(19) United States
(12) Patent Application Publication
(10) Pub. No.: US 2011/0135749 A1
(43) Pub. Date: Jun. 9, 2011

(54) LOW SHRINKAGE MULTIFUNCTIONAL SSQ RESINS

(75) Inventors: Alan Sellinger, Singapore (SG); Mui Siang Soh, Singapore (SG); U Jin Adrian Yap, Singapore (SG)

(73) Assignee: AGENCY FOR SCIENCE, TECHNOLOGY AND RESEARCH, (SG)

(21) Appl. No.: 12/920,463

(22) PCT Filed: Mar. 4, 2008
(86) PCT No.: PCT/SG08/00073

§ 371 Date: Feb. 7, 2011

(51) Int. Cl. A61K 33/42; A61K 31/155; A61K 33/08; A61K 33/14; A61K 33/24; A61P 1/02; A61P 31/04; A61K 6/08; A61C 5/00

(52) U.S. Cl. 424/601; 514/635; 424/602; 424/693; 424/676; 424/673; 424/650; 523/116; 433/217.1

(57) ABSTRACT

The present invention relates to low shrinkage multi functionalized silsesquioxane (SSQ) derivatives, to nanocomposite materials comprising these SSQ's and to the use of the nanocomposite material as a polymerizable resin in biologically compatible materials.

### FIELD OF THE INVENTION

The present invention relates to low shrinkage multi functionalized silsesquioxane (SSQ) derivatives, to nanocomposite materials comprising these SSQ's and to the use of the nanocomposite material as a polymerizable resin in biologically compatible materials.

### BACKGROUND

Dental composites which revolutionized the practice of clinical dentistry in the 1960s are complex, tooth-colored filling materials composed of synthetic polymers, inorganic fillers, initiators, activators, stabilizer and silane coupling agents which bond the reinforcing fillers to the polymer matrix. Since their introduction, they have found wide applications as anterior and posterior restorations, indirect inlays/onlays, pit and fissure sealants and wear-resistant denture teeth.¹ Despite their popularity and remarkable improvements in formulation, materials and curing technology, the currently available composite materials still have the inherent disadvantage of polymerization shrinkage and its accompanying stress.

All known dental composites exhibit 2-14% volumetric shrinkage during the polymerization process.²⁻⁶ When composite shrink, stress are generated at the composite/tooth interface. These shrinkage stresses can cause marginal openings if the bonding system is unable to withstand the polymerization forces and thus lead to leakage and ultimately caries. Despite the dramatic improvements in the formulation of newer generation bonding agents with enhanced marginal adaptation and bond strengths, a perfect marginal seal is still not achievable. Clinical studies carried out for resin-based composite restorations for Class I and II cavities for a period of 3 to 6 years have also shown that secondary and/or recurrent caries were the main reasons for restoration failure and polymerization shrinkage has been cited as one of the most significant factor influencing the seal between tooth structure and polymer-based restorative materials. Thus, the major and most significant drawback of composite-based resins is the shrinkage during the polymerization process.

The total shrinkage of composite materials can be divided into pre-gel and post-gel phases. During the pre-gel polymerization, the composite is able to flow and stresses within the structure are relieved.⁷ After gelation, viscosity increases significantly and stresses due to shrinkage cannot be compensated. Post-gel polymerization thus results in significant stresses in the surrounding tooth structure and composite tooth bond that may lead to bond failure, microleakage, post-operative sensitivity, pulp pathoses and recurrent caries.⁸⁻¹¹ These stresses could also produce defects at the composite/tooth interface causing the tooth to fracture.¹² Therefore, reduction in polymerization shrinkage of dental composites has become a major focus in the development of new monomers for dentistry.

In order to overcome the problem on polymerization shrinkage and its associated stresses, a few systems have been explored. For example, ring opening monomers such as spiro-orthocarbonate (SOCs)¹³ that expand during polymerization through a double-ring opening process, acrylates and methacrylates containing spiro ortho esthers¹⁴ that are capable of being polymerized by heat, ionic and free radical initiators, oxetanes,¹⁵ oxybismethacrylates¹⁶ and highly branched methacrylates¹⁷ have been developed. Although these polymers are promising, problems balancing mechanical properties, water sorption, solubility, curing times and expansion still exist.

One recent approach in the development of dental restorative composites has been in the area of organic-inorganic hybrid nanocomposite materials.^{18,19} For example, a class of fused cyclic siloxane caged materials termed silsesquioxanes (SSQ), commonly known as Polyhedral Oligomeric Silsesquioxanes (POSS™), are known. The octameric SSQ with a rigid framework resembling that of silica is a unique class of inorganic/organic hybrid materials with a diameter of 0.53 nm. Its versatile chemical reactivity not only allows the development of a wide range of hybrid materials with many appealing properties²⁰⁻²⁶ but has also found numerous applications in photoresists, abrasion resistant coatings, liquid display elements, insulating layers on electronic circuit boards and optical fiber coatings.²⁵⁻²⁶ The incorporation of these nanosized molecules also offer the advantage of improved solubility, adhesion, copolymerization, grafting and blending.^{18,27}

However, still there is much development desired for obtaining SSQ compounds having improved stability, crosslink density and packing. In particular, there remains the need in the art for dental resin materials that have minimal shrinkage without sacrificing other advantages physical properties.

### SUMMARY

The objective of the present invention was to synthesize and provide low-shrinking multifunctional silsesquioxanes (SSQ).

In a first aspect, the present invention provides multi functionalized SSQ derivative according to formula (I)

[((R¹)ₐ(R²)_{b}(R³)_{c}(R⁴)_{d})((R⁵)ₙSiₙO_{z})] (I)

wherein

n is an integer from 6 to 14;

z is 1.5n or 2.5n;

R¹, R², R³, and R⁴ are independently selected from the group of polymerizable residues consisting of C₂-C₂₀ alkene, C₂-C₂₀ alkyne, allyl, allyl glycidyl ether, C₂-C₂₀ alkylalkene, alkylalkynes, acrylates, methacrylates, benzoxazines, epoxides and oxetanes;

R⁵ is independently selected from (C(R^{I})(R^{II}))_{q}, ((C(R^{I})(R^{II})_{q}N(R^{III})), [(CH₂)ₘO]ᵣ and [(Si(R^{IV})₂O)]ₛ, wherein R^{I}, R^{II}, R^{III} and R^{IV} are independently selected from hydrogen, C₁-C₂₀ alkyl, and C₆-C₁₀ aryl; m is an integer from 1 to 10; and q, r and s are independently an integer from 0 to 10;

a, b, c and d are independently an integer from 0 to n, wherein a+b+c+d=n,

with the proviso that in case z is 1.5n than at least one of R¹, R², R³ and R⁴ is not (CH₂)₃OC(O)C(CH₂)(CH₃), (CH₂)₃OC(O)C(CH₂)(H), CH(CH₂),

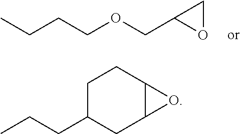

In a second aspect, the present invention provides a nanocomposite material comprising
polymerizable monomers comprising at least one multi functionalized silesquioxane (SSQ) derivative of the present invention as co-monomer; and
a curing system.

In a further aspect, the present invention provides the use of the nanocomposite material of the present invention as a polymerizable resin for biologically compatible materials.

### BRIEF DESCRIPTION OF THE DRAWINGS

FIG. 1 illustrates two general preparation examples of the SSQ compounds of the present invention.

FIG. 2 illustrates an example of a general reaction scheme for the preparation of one of the SSQ compounds of the present invention having a cage with 8 Si atoms.

FIG. 3 illustrates a further example of the general preparation scheme for an exemplary compound of the present invention having 8 Si atoms.

FIG. 4 illustrates SSQ compounds of the present invention having allyl ether methacrylate, propargyl methacrylate and vinyl cyclohexene epoxide substituents.

FIG. 5 illustrates four SSQ compounds of the present invention.

FIG. 6 illustrates a graph showing the mean shrinkage of SSQ compounds of the present invention and compositions of the prior art during light curing.

FIG. 7 illustrates a graph showing the mean shrinkage of SSQ compounds of the present invention and compositions of the prior art post-light polymerization.

FIG. 8 illustrates a graph showing the mean hardness of SSQ compounds of the present invention and compositions of the prior.

FIG. 9 illustrates a graph showing the mean modulus of SSQ compounds of the present invention and compositions of the prior.

FIG. 10 illustrates a graph showing the depth of cure of SSQ compounds of the present invention and compositions of the prior.

FIG. 11 illustrates a graph showing the mean water sorption obtained for SSQ compounds of the present invention and compositions of the prior.

FIG. 12 illustrates the polymerization of epoxy groups using a 3 component initiating system.

FIG. 13 illustrates an example of a general reaction scheme for the preparation of (HSiMe₂OSiO_{1.5})₈.

### DETAILED DESCRIPTION OF THE INVENTION

In the following description non-limiting embodiments of the process of the invention will be explained.

According to the present invention, it has been surprisingly found that a new class of multi functionalized silsesquioxane (SSQ) derivatives may be easily synthesized, wherein said compounds may be used as low-shrinking monomers for biologically compatible materials, for example for dental applications. The compounds have been isolated in high yields and form highly reactive, viscous liquids that can be polymerized both thermally and/or photochemically to produce clear hybrid polymers. It has unexpectedly been discovered that the SSQ of the present invention show improved low shrinkage upon curing, together with improved fracture resistance.

In the context of the present invention, the term “comprising” or “comprises” means including, but not limited to, whatever follows the word “comprising”. Thus, the use of the term “comprising” indicates that the listed elements are required or mandatory, but that other elements are optional and may or may not be present.

The following terms refer to any groups mentioned in the present invention unless otherwise indicated.

The term “alkyl”, alone or in combination, refers to a fully saturated aliphatic hydrocarbon. In certain embodiments, alkyls are optionally substituted. In certain embodiments, an alkyl comprises 1 to 30 carbon atoms, for example 1 to 20 carbon atoms, wherein (whenever it appears herein in any of the definitions given below) a numerical range, such as “1 to 20” or “C₁-C₂₀”, refers to each integer in the given range, e.g. “C₁-C₂₀ alkyl” means that for example an alkyl group comprising only 1 carbon atom, 2 carbon atoms, 3 carbon atoms, etc., up to and including 20 carbon atoms. Examples of alkyl groups include, but are not limited to, methyl, ethyl, n-propyl, isopropyl, n-butyl, isobutyl, sec-butyl, tert-butyl, tert-amyl, pentyl, hexyl, heptyl, octyl and the like.

The term “alkene”, alone or in combination, refers to an aliphatic hydrocarbon having one or more carbon-carbon double-bonds. In certain embodiments, alkene groups are optionally substituted. Examples of alkene groups include, but are not limited to, ethenyl, propenyl, butenyl, 1,4-butadienyl, pentenyl, hexenyl, 4-methylhex-1-enyl, 4-ethyl-2-methylhex-1-enyl and the like.

The term “alkyne”, alone or in combination, refers to an aliphatic hydrocarbon having one or more carbon-carbon triple-bonds. In certain embodiments, alkyne groups are optionally substituted. Examples of alkyne groups include, but are not limited to, ethynyl, propynyl, butynyl, and the like.

The term “alkylalkene” refers to a group comprising an alkyl group bound to an alkene group. In certain embodiments, arylalkenes are optionally substituted. Examples of alkylalkene include, but are not limited to, (CH₂)_{w}—CH(CH₂), (CH₂)_{w}—C(CH₃)(CH₂), wherein w is an integer from 1 to 15, and the like.

The term “alkylalkyne” refers to a group comprising an alkyl group bound to an alkyne group. In certain embodiments, arylalkynes are optionally substituted. Examples of alkylalkynes include, but are not limited to, (CH₂)_{w}—C(CH), wherein w is an integer from 1 to 15, and the like.

The term “acrylate” refers to a group of formula X—OC(O)C(CH₂)(H). X may be C₁-C₂₄ alkyl, [(CH₂)ₘO]ᵣ, C₂-C₁₂ ether, vinyl group or allyl group, wherein r is an integer from 1 to 10. Examples of acrylates include, but are not limited to, acrylate, propargyl acrylate, allyl acrylate, 2-allyloxyethyl acrylate, 2-propargyloxyethyl acrylate, 1-hexenylacrylate, and the like.

The term “methacrylate” refers to a group of formula X—OC(O)C(CH₂)(CH₃). X may be C₁-C₂₄ alkyl, [(CH₂)ₘO]ᵣ, C₂-C₁₂ ether, vinyl group or allyl group, wherein r is an integer from 1 to 10. Examples of methacrylates include, but are not limited to, di(propylene glycol) allyl ether methacrylate, propargyl methacrylate, 2-(methacryloyloxy)ethyl ester, allyl methacrylate, allyl acrylate, propargyl methacrylate, propargyl acrylate, and the like.

The term “epoxide” refers to compounds having one or more epoxide (oxirane) functionalities, and includes monomeric epoxy compounds and epoxides of the oligomeric or polymeric type, which can be aliphatic, cycloaliphatic, aromatic, or heterocyclic. The epoxides may vary from low molecular weight monomeric materials to oligomers to high molecular weight polymers and may vary greatly in the nature of their backbone and substituent groups. Examples of epoxides include, but are not limited to,

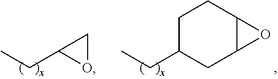

wherein x is an integer from 1 to 10, 3,4-epoxycyclohexylmethyl-3,4-epoxycyclohexanecarboxylate, 3,4-epoxy-2-methylcyclohexylmethyl-3,4-epoxy-2-methylcyclohexane carboxylate, bis(3,4-epoxy-6-methylcyclohexylmethyl) adipate, 4-vinyl-cyclohexane epoxide, and the like.

The term “optionally substituted” refers to a group in which none, one, or more than one of the hydrogen atoms has been replaced with one or more group(s) are independently selected from: alkyl, aryl, arylalkyl, hydroxy, alkoxy, halo, carbonyl, nitro, silyl, and amino, including mono- and di-substituted amino groups. In embodiments in which two or more hydrogen atoms have been substituted, the substituent groups may be linked to form a ring.

In one embodiment the present invention is directed to multi functionalized silsesquioxane (SSQ) according to formula (I)

[((R¹)ₐ(R²)_{b}(R³)_{c}(R⁴)_{d})((R⁵)ₙSiₙO_{z})] (I)

wherein

n is an integer from 6 to 14;

z is 1.5n or 2.5n;

R¹, R², R³, and R⁴ are independently selected from the group consisting of C₂-C₂₀ alkene, C₂-C₂₀ alkyne, allyl, allyl glycidyl ether, C₂-C₈ alkylalkene, alkylalkynes, acrylates, methacrylates, benzoxazines, epoxides and oxetanes;

R⁵ is independently selected from (C(R^{I})(R^{II}))_{q}, ((C(R^{I})(R^{II})_{q}N(R^{III})), [(CH₂)ₘO]ᵣ and [(Si(R^{IV})₂O)]ₛ, wherein R^{I}, R^{II}, R^{III} and R^{IV} are independently selected from hydrogen, C₁-C₂₀ alkyl, and C₆-C₁₀ aryl; m is an integer from 1 to 10; and q, r and s are independently an integer from 0 to 10;

a, b, c and d are independently an integer from 0 to n, wherein a+b+c+d=n,

with the proviso that in case z is 1.5n than at least one of R¹, R², R³ and R⁴ is not (CH₂)₃OC(O)C(CH₂)(CH₃), (CH₂)₃OC(O)C(CH₂)(H), CH(CH₂),

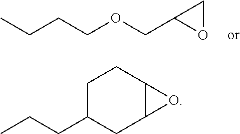

In the above formula (I), the basic silsesquioxane structure is presented by (SiₙO_{z}). In case of z=1.5n, each silicon atom therein is bound to an average of one and a half (sesqui) oxygen atoms. In one embodiment of the present invention, an additional oxygen atom is linked to each respective Si-atom of the cage resulting in a general formula of (SiₙO_{2.5n}), i.e. in the above formula z=2.5n. To the latter oxygen atom and to the Si-atom in the formula (SiₙO_{1.5n}) the (bridging) group R⁵ is bound. The silsesquioxane molecules have rigid, thermally stable silicon-oxygen frameworks. Depending on the number of n, the silsesquioxane forms a 3-dimensional cage which is then optionally substituted at the respective Si-atoms.

n is an integer from 6 to 14, i.e. for n=6 a cage with six Si-atoms is formed, wherein, for example, with n=8, an octahedral cage is obtained. n may be any integer from 6 to 14, i.e. n may be 6, 7, 8, 9, 10, 11, 12, 13 or 14. In one embodiment n is an even number of 6, 8, 10, 12 or 14. In one further embodiment n=8.

As already stated above and as indicated in formula (I), each Si atom (or Si—O-Group in case of the general formula (SiₙO_{2.5n})) of the SSQ is substituted with a R⁵ substituent. R⁵ is a bridging group (or spacer group) which is further substituted by one of the polymerizable residues R¹, R², R³, and R⁴. R⁵ may be, but is not limited to, (C(R^{I})(R^{II}))_{q}, ((C(R^{I})(R^{II})_{q}N(R^{III})), [(CH₂)ₘO]ᵣ and [(Si(R^{IV})₂O)]ₛ. Examples of R⁵ may be, but are not limited to, CH₂, CH₂—CH₂, CH₂—CH₂—CH₂, CH₂O, CH₂—CH₂O, CH₂NH, CH₂CH₂NH, Si(H₂)O, Si(CH₃)₂O, and the like.

R¹, R², R³, and R⁴, which are polymerizable residues, are connected via R⁵ to the SSQ cage. These substituent groups will have at least one polymerizable function, for example at the end which is not bound to the SSQ cage. In one embodiment of the present invention, examples of R¹, R², R³, and R⁴ may be, but are not limited to, C₂-C₂₀ alkene, C₂-C₂₀ alkyne, allyl, allyl glycidyl ether, C₂-C₂₀ alkylalkene, alkylalkynes, acrylates, methacrylates, benzoxazines, epoxides, oxetanes and the like. Each of R¹, R², R³, and R⁴ may be present in the functionalized SSQ of the present invention, wherein they may be the same or different. It is also possible that only one, two or three of R¹, R², R³, and R⁴ may be present in the functionalized SSQ. The actual number of each of R¹, R², R³, and R⁴ is given by the subscript a, b, c and d, which is an integer from 0 to n, wherein a+b+c+d=n. This means that every Si atom of the SSQ cage is substituted with one of the polymerizable residues. For example, in one embodiment, a may be n. In another embodiment, a may be n−2 and b may be 2 or a and b may be the same, for example each a and b may be 4 in case n=8. Each possible combination of the substituents R¹, R², R³, and R⁴ is suitable in the present invention.

In one embodiment of the present invention, a+b=n (i.e. c+d=0). In this case, for example, R¹ and R² may be independently selected from di(propylene glycol) allyl ether methacrylate, 4-vinyl-cyclohexene epoxide and propargyl methacrylate.

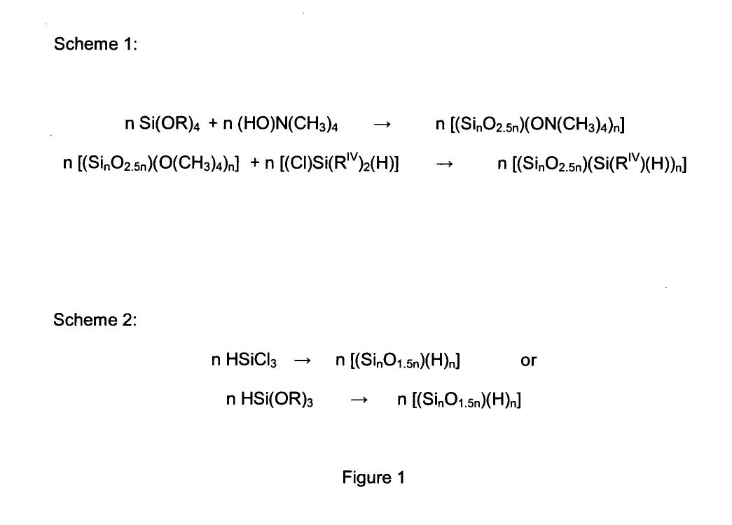

Examples of the general preparation methods for the precursor SSQ compounds of the present invention can be taken from FIGS. 1 to 3. Basically, two main preparation procedures can be mentioned. Method 1 is illustrated in Scheme 1 of FIG. 1. A tetraalkyloxysilane is reacted with a tetraalkylammonium salt, such as for example, but not limited to, tetramethylammonium hydroxide in a water/alcohol mixture. A 1:1:10 M solution of “Si”:“N”:“H₂O” may be prepared respectively. However, further ratios of the compounds may be suitable in the present invention. Depending on the reaction conditions, a SSQ cage having 6 to 14 Si atoms can be obtained, wherein each Si atom is substituted with a (ON(CH₃)₄) group. This SSQ cage is further reacted with, for example, a suitable silane compound. For example, a substituted chlorosilane such as dimethylchlorosilane, may be used to obtain the respective substituted SSQ cage.

In Scheme 2 of FIG. 1 a further example for the preparation of the precursor SSQ cages is illustrated. The method described in Scheme 2 is making use of a more corrosive monomer and volatile as the melting point is just above room temperature. In this particular example, trichlorosilane (HSiCl₃) or (HSi(OR)₃) is reacted using a catalyst system to prepare the respective SSQ cage having a hydrogen as substituent on every Si atom.

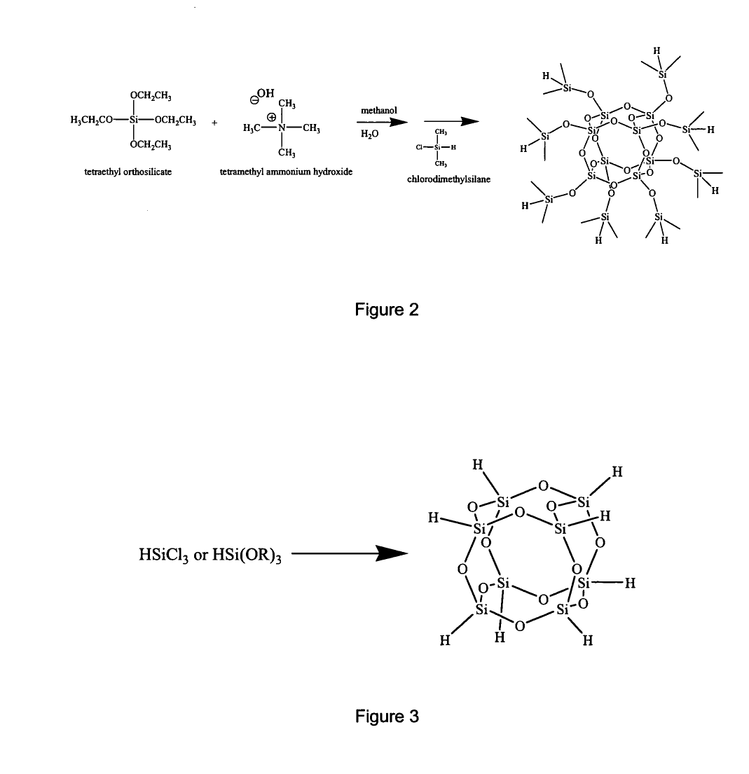

FIGS. 2 and 3 are specific examples of the preparation routes described above, wherein n=8.

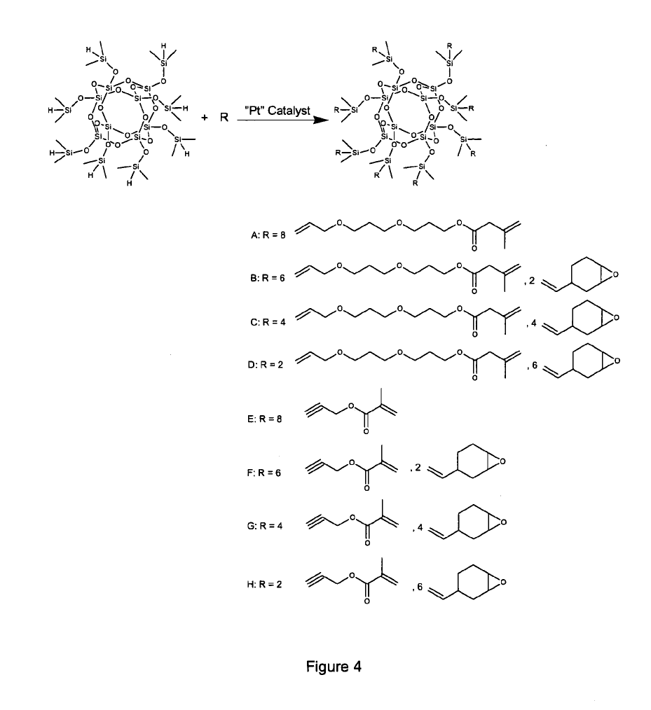

FIG. 4 describes a possible synthesis route for linking the polymerizable moieties to the precursor SSQ cage. The compounds reacted with the precursor SSQ cage should be bifunctional. “bifunctional” in this case means that these compounds should have one functional group capable of reacting with the precursor SSQ cage and another functional group which may be polymerizable. For example, (substituted) acrylates, methacrylates and epoxides may be reacted with the precursor SSQ cage. In this reaction step, a suitable catalyst such as a Pt catalyst may be used. For example, platinum divinyltetramethyldisiloxane [Pt(dvs)], platinum dicyclopentadiene [Pt(dcp)], hexachloroplatinic acid (H₂PtCl₆) and the like may be used, but is not limited to. Illustrative examples for this preparation method are given below.

Various novel low shrinking organic/inorganic hybrid materials based on functionalized SSQ were successfully synthesized and characterized. For example, methacrylate and epoxy substituents have been obtained. Thus, compounds with different functional groups per SSQ cage were made possible in this invention. All compounds were obtained in high yields (>80%) and were synthesized based on inexpensive starting materials. The compounds obtained were viscous liquids at room temperature and contained up to about 48% masked silica making them ideal for, for example, dental materials. With this invention, novel low shrinking fully functionalized SSQ resins with different functional groups ratio were obtained.

A further aspect of the present invention is directed to a nanocomposite material. The definition of nanocomposite material encompasses a large variety of systems such as one-dimensional, two-dimensional, three-dimensional and amorphous materials, made of distinctly dissimilar components and mixed at the nanometer scale. These materials may be created by introducing nanoparticulates into a macroscopic sample material. After adding nanoparticulates to the macroscopic sample material, the resulting nanocomposite may exhibit drastically enhanced properties. For example, it may result in enhanced mechanical properties such as stiffness and strength.

The nanocomposite material of the present invention comprises polymerizable monomers comprising at least one of the multi functionalized silsesquioxane (SSQ) derivative described above as co-monomer and a curing system.

The polymerizable monomers of the nanocomposite material comprise at least one of the multi functionalized silsesquioxane (SSQ) derivative of the present invention and at least one further monomer which may be, but is not limited to, bisphenylglycidyl methacrylate (Bis-GMA), triethylene glycol dimethacrylate (TEGDMA), urethane dimethacrylate (UDMA), ethoxylated bisphenol-A-dimethacrylate (bis-EMA), decanediol dimethacrylate (D₃MA), urethane tetramethacrylate (UTMA), methyl methacrylate, 2-hydroxyethyl methacrylate, hexandiol methacrylate, dodecanediol dimethacrylate, bisphenol-A-dimethacrylate, 2,6-di-tert-butyl-4-metyhlphenol (BHT), 2-hydroxyethylmethacrylate (HEMA), N,N-dimethyl-p-toluidine or mixtures thereof.

The nanocomposite material according to the present invention may contain a curing system. The curing system assists in the polymerization of the nanocomposite material. The curing system may include, but is not limited to, at least one of polymerization initiators, polymerization accelerators, stabilizers, ultraviolet light absorbers, antioxidants and other additives known in the art. The amount of the respective compounds of the curing system may be, but is not limited to, about 0.1 to about 5 wt % based on the total nanocomposite material. In one embodiment of the present invention, the curing system may be used in an amount of about 0.5 to about 3 wt %, such as about 1 wt %.

Suitable polymerization initiators are those initiators, which can be utilized in UV-activated cure or visible light-activated cure compositions. For example, visible light curable compositions employ light-sensitive compounds, including but not being limited to benzil, benzoin, benzoin methyl ether, DL-camphorquinone (CQ), and benzil diketones. Either UV-activated cure or visible light-activated cure (approximately 230 to 750 nm) is acceptable. The amount of photoinitiator is selected according to the curing rate desired. A minimal catalytically effective amount is generally about 0.01 wt % of the total resin compositions, and will lead to a slower cure. Faster rates of cure are achieved with amounts of catalyst in the range from greater than about 0.01 percent to about 5 wt % of the total material.

The polymerization accelerator may be, but is not limited to, an amine, ammonia, an acid or the like. For example, the various organic tertiary amines well known in the art may be used. In visible light curable dental composite materials, the tertiary amines are generally acrylate derivatives such as in an amount of about 0.05 to about 5.0 wt % of the total material. In one embodiment of the present invention the amine may be a tertiary amine, such as, but not limited to, N(C₂H₅)₃, N(C₆H₅)₃, ethyl 4-(dimethylamino)benzoate (EDMAB), 2-[4-(dimethylamino)phenyl]ethanol, N,N-dimethyl-p-toluidine (DMPT), bis(hydroxyethyl)-p-toluidine, dimethylaminoethyl methacrylate, diethylaminoethyl methacrylate (DEAEMA) or the like.

The stabilizers may be, but is not limited to, hydroquinone, hydroquinone monomethyl ether, t-butyl paracresol, hydroxyl methoxybenzophenone and the like.

The ultraviolet light absorbers may be used in an amount of about 0.05 to about 5.0 wt % of the total nanocomposite material. Such UV absorbers are particularly desirable in the visible light curable dental restorative materials in order to avoid discoloration of the resin from incident ultraviolet light. Suitable UV absorbers may be benzophenones and the like.

In one embodiment the nanocomposite material according to the present invention may additionally comprise at least one filler. The at least one filler may comprise one or more of inorganic fillers. For example, fillers may include those that are capable of being covalently bonded to the resin matrix itself or to a coupling agent that is covalently bonded to both. Examples of suitable filling materials include, but are not limited to, silica, quartz, silica glass, strontium silicate, strontium borosilicate, lithium silicate, lithium alumina silicate, amorphous silica, ammoniated or deammoniated calcium phosphate, tricalcium phosphate alumina, zirconia, tin oxide, titania, apatites, hydroxyapatites, modified hydroxyapatite compositions, bismuth oxide, barium sulfate, bismuth subcarbonate or the like. The fillers may have a particle size in the range from about 0.1 to about 5.0 microns.

The silica glass may comprise strontium, barium, zinc, boron, yttrium, aluminoborosilicate glass, strontium-alumino-fluoro-silicate glass or colloidal glass or other fluoride releasing glasses.

The amount of the total filler in the nanocomposite material may vary widely, being in the range from about 1 to about 90 wt % of the total composition. The amount used is determined by the requirements of the particular application of the nanocomposite material. Thus, when used as a dental material, for example a crown and bridge material, it generally comprises from about 60 to about 90 wt % filler; luting cements comprise from about 20 to about 80 wt % filler; sealants generally comprise from about 1 to about 20 wt % filler; adhesives generally comprise from about 1 to about 30 wt % filler; and restorative materials comprise from about 50 to about 90 wt % filler, with the remainder in all cases being the resin composition and curing system. In this respect it should be noted that the amount of the fillers depends on the size and types of fillers and resins and can empirically be determined by the person skilled in the art, if desired. In some illustrative embodiments the filler may be used in an amount of about 1 to about 90 wt % of the nanocomposite material, for example in an amount of about 10 to about 80 wt %. In one embodiment, the amount of the filler may be from about 25 to about 70 wt %, such as about 63%.

In a further embodiment of the present invention, the nanocomposite material may additionally comprise a therapeutic agent. This therapeutic agent may be described as an agent that is suitable in providing a beneficial therapeutic effect in healing or treating dental diseases. For example, the therapeutic agent may be an antibacterial and/or a remineralization (enhancing) agent. Illustrative examples of remineralization (enhancing) agents include calcium compounds such as calcium phosphate or calcium hydroxide, fluorine compounds such as sodium fluoride, potassium fluoride, ammonium fluoride, stannous fluoride, calcium fluoride, sodium or potassium monofluorophosphate or zinc compounds such zinc citrate, zinc oxide or zinc stearate. Such remineralization (enhancing) compounds are described in U.S. Pat. No. 7,300,645, for example. Illustrative examples of antibacterial agents, include but are not limited, to chlorohexidine, or an halogenated diphenyl ether or phenolic antibacterial compound such as tricosan (see U.S. Pat. No. 6,451,291, and the like.

The multifunctional SSQ of the present invention may be used in an amount of about 1 to about 90 wt % based on the total amount of the nanocomposite material. In one embodiment, the SSQ is used in an amount of about 3 to about 50 wt % based on the total amount of the nanocomposite material, for example in an amount of about 5 wt % to about 40 wt %, for example about 20 wt % based on the total amount of the nanocomposite material. The amount actually used in the nanocomposite material may depend on the type of Si cage, i.e. the SSQ resin and can be determined empirically, if desired, by the person skilled in the art. The above described nanocomposite material is a low-shrinking material. This means that upon polymerization the shrinkage of the material is insignificant low compared to the shrinkage properties of the materials of the prior art. The degree of shrinkage can be provided in terms of linear percent shrinkage. Polymerization shrinkage of composite occurs by bond formation between monomers during polymerization. The distance between monomers due to van der waals' forces is transformed into the distance of covalent bonds of the polymer that are formed. Magnitude of shrinkage is determined by the number of covalent bonds formed and the size of monomers. Linear Percent shrinkage measures post-gel changes in the linear dimension.

In one embodiment the material has a linear percent shrinkage below about 4.0, for example below about 2.0. In one embodiment of the present invention the linear percent shrinkage may be about 0.25 to about 0.5, for example about 0.28 to about 0.4. The linear percent shrinkage may also be calculated as volumetric percent shrinkage using, for example, the formula described in Julita Jakubiak & Lars Ake Linden, Polimery (2001), 46(7-8), 522-528.

All essential and optional materials of the nanocomposite material may be mixed at the same time or subsequently. In one manner of proceeding the polymerizable monomers and the SSQ and any additional filler are mixed. The optional curing system is added to the mixture, which is again stirred and stored or used, as appropriate. The cure may be initiated through the use of UV light or by raising the temperature of the mixture. In another embodiment of the present invention the polymerizable monomers, SSQ, light initiators and activators are mixed followed by mixing of the fillers. Subsequently, the cure may be initiated through the use of blue visible light curing units.

The nanocomposite material of the present invention may be used as a polymerizable resin for biologically compatible materials. Biologically compatible materials may be, for example, dental materials. Dental materials are materials used to restore diseased or damaged teeth to health and function. The corrosive nature of saliva and the expansion and contraction of tooth structure with changes in temperature make great demands upon a dental material. The stress brought to bear on the restoration by masticatory forces also makes great demands. Dental materials must be compatible with living tissue. If used in the anterior region of the mouth, the materials must also be esthetically pleasing. Dentals materials, used when and where indicated, help to ensure the placement of a successful restoration and preservation of the tooth.

All the above properties are met by the nanocomposite material of the present invention. The materials have improved strength and in particular shrinkage properties compared to conventional dental materials known in the prior art. Further, the dental materials may be formulated to have additional improved properties, such as water sorption or bonding to tooth substrates as expressed in measured shear bond. The nanocomposite material may be applied to a site of a tooth and subsequently cured by any conventional means. The nanocomposite material may also be used as coatings, lenses and plastics. In one embodiment, the low shrinking nanocomposite material may be used as a direct restorative material.

The substituent groups are chosen to be consistent with the current chemistry used in commercial dental composites, while the SSQ are introduced to provide a rigid three-dimensional chemical platform and “masked silica” source.

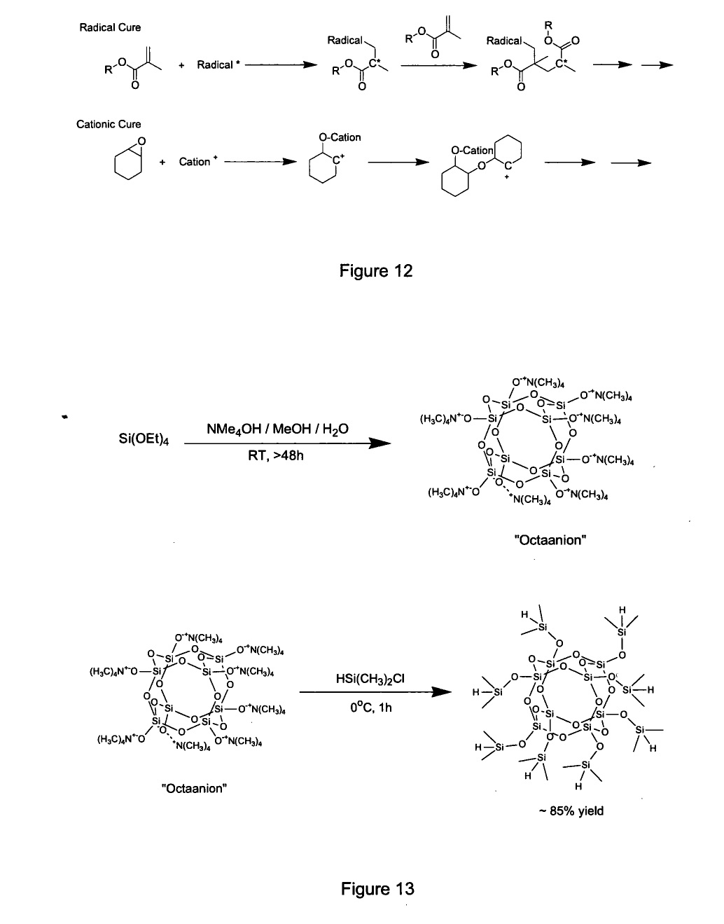

The cationic initiators can be incorporated to the nanocomposite to allow for a dual cure system where the epoxy ring can be opened to compensate for volumetric shrinkage for the multi-functionalized SSQ. The epoxy groups on SSQ may be opened using, for example, a three component initiating system comprising of CQ, an iodonium salt and an electron donor (DMAEMA) using the current light curing units (FIG. 12).

As will be demonstrated in the examples, polymerization shrinkage associated with all SSQ compounds was found to be significantly lower than the control materials (Bis-GMA/TEGDMA). In order to obtain a material with low shrinkage and comparable mechanical properties, effects of SSQ compounds as copolymers were also evaluated. The developed SSQ resins which were liquids at room temperature do not require significant changes in processing. SSQ resins can be simply mixed and copolymerized by traditional methods. They form true molecular dispersions when mixed into polymer formulations with no phase separation. The inclusion of SSQ resins into polymer formulations can also lead to dramatic improvements in polymer properties.

### EXAMPLES

The following experimental examples are provided to further illustrate the present invention and are not intended to be limiting to the scope of the invention.

¹H, ¹³C and ²⁹Si NMR solution spectra were run in CDCl₃ in 5 mm diameter tubes and recorded using a Bruker DRX 400 MHz NMR spectrometer. Chemical shifts were assigned using residual CHCl₃ (¹H, ¹³C) and TMS (²⁹Si) as an internal standard. Fourier Transformation Infrared (FTIR) spectra were recorded on a Perkin Elmer Spectrum 2000 FTIR spectrophotometer with a resolution of 4 cm⁻¹. Liquid samples were cast on NaCl plates and solid samples were pressed into KBr pellets. A minimum of 32 scans were collected for each sample.

### Example 1

### General procedure for preparation of (HSiMe₂OSiO_{1.5})₈

Tetraethylorthosilicate (SiOEt)₄ was reacted with tetramethylammonium hydroxide (25% in methanol) (NMe₄OH) in methanol/water (MeOH/H₂O) solution to make a 1:1:10 M solution in “Si”:“N”:“H₂O” respectively. After stirring vigorously for greater than 48 h at room temperature, a quantitative yield of the “octaanion” was obtained. Reaction of (HSiMe₂OSiO_{1.5})₈ was then carried out by adding solution of the “octaanion” dropwise to an ice-cold solution containing chlorodimethylsilane (HSi(CH₃)₂Cl) (6 equivalents per O⁻) in hexane with vigorous stirring for 1 h. White solid in yields of ˜85% was obtained upon solvent removal. The resultant solid was then purified by rinsing with cold methanol. The product is soluble in dichloromethane (CH₂Cl₂), THF (tetrahydrofuran), CHCl₃, and non-polar solvents such as hexane, pentane and toluene. The obtained silsesquioxane core was then used for subsequent synthesis.

### Example 2

### Pt-Catalyzed reaction of Di(propylene glycol) allyl ether methacrylate with Octakis(dimethylsiloxy)silsesquioxane

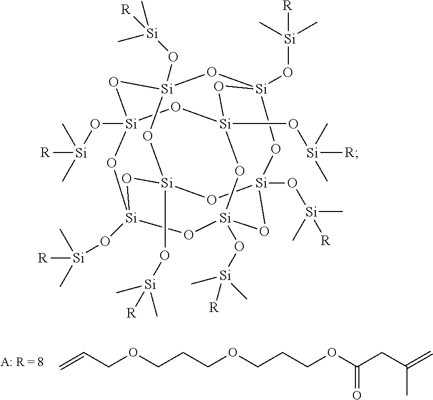

Reaction A was carried out by adding (HSiMe₂OSiO_{1.5})₈ (1.0 g, 0.98 mmol) and anhydrous toluene (10 ml) to a 50 ml round bottom flask equipped with a stir bar and air condenser under Ar (FIG. 3). The mixture was stirred for 5 min to allow for all (HSiMe₂OSiO_{1.5})₈ to dissolve. Di(propylene glycol)allyl ether methacrylate (2.14 g, 8.83 mmol) followed by Pt(dvs) (0.01 ml of 2 mmol solution in anhydrous xylene) were then added dropwise into the flask. The mixture was allowed to stir at 55° C. for 24 h. Reaction completion was monitored by the disappearance of the SiH absorption peak at 2145 cm⁻¹ using FTIR. Small amount of excess reactant and solvent were removed effectively by vacuum evaporation technique using a high vacuum pump. A viscous liquid with a product yield of 95% was obtained. ¹H NMR (400 MHz, CDCl₃) (δ, ppm): 6.08, 5.52 (d, 16H, CH₂═CCH₃(CO)), 3.20-3.70 (m, 56H, OCH₂), 1.92 (s, 24H, CH₂═CCH₃), 1.0-1.4 (m, 48H, CH₂CH₂O), 0.56-0.60 (m, 16H, (CH₃)₂SiCH₂), 0.12 (s, 48H, (CH₃)₂SiCH₂). ¹³C NMR (100 MHz, CDCl₃) (δ, ppm): 166.81 (C═O), 136.63 (CH₃C═CH₂), 125.10 (CH₃C═CH₂), 76.72-69.77 (OCH₂), 23.10 (OCH₂CH₂CH₂O), 18.22 (CH₃C═CH₂), 17.31-16.20 (CH₂CH₂CH₂O), 13.65 (Si(CH₃)₂CH₂), −0.44 (Si(CH₃)₂). ²⁹Si NMR (79 MHz, CDCl₃) (δ, ppm): 13.0 (Si(CH₃)₂), −108.8 (SiO₄). FTIR (cm⁻¹): 1718 (C═O), 1638 (C═C), 1254 (Si(CH₃)₂), 1094 (Si—O—Si).

### Example 3

### Pt-Catalyzed reaction of Di(propylene glycol) allyl ether methacrylate and 4-vinyl-cyclohexene epoxide with Octakis(dimethylsiloxy)silsesquioxane

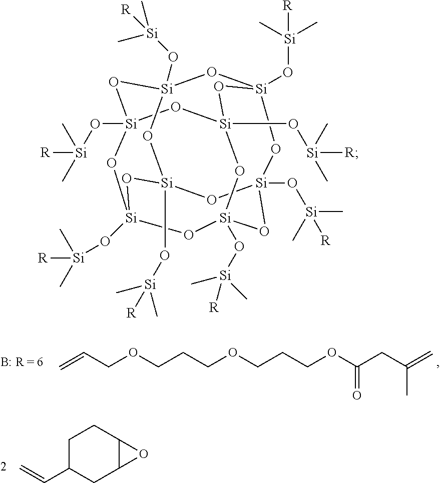

Reaction B was carried out in two steps by adding (HSiMe₂OSiO_{1.5})₈ (1.0 g, 0.98 mmol) and anhydrous toluene (10 ml) to a 50 ml round bottom flask equipped with a stir bar and condenser under Ar. The mixture was stirred for 5 mins to allow for all (HSiMe₂OSiO_{1.5})₈ to dissolve. 4-vinyl-cyclohexene epoxide (0.24 g, 1.96 mmol, 2 equivalents) followed by Pt(dcp) (0.01 ml of 2 mmol solution in anhydrous xylene) were then added dropwise into the flask. The mixture was allowed to stir at 55° C. for 24 h. Di(propylene glycol)allyl ether methacrylate (1.43 g, 5.90 mmol, 6 equivalents) was then added dropwise into the reaction and allowed to stir for another 24 h to complete the reaction. Reaction completion was monitored by the disappearance of the SiH absorption peak at 2145 cm⁻¹ using FTIR. Small amount of excess reactant and solvent were removed effectively by vacuum evaporation technique using a high vacuum pump. A viscous liquid with a product yield of 88% was obtained. The above synthetic procedures were repeated for the synthesis of compound C and D. Compound B: ¹H NMR (400 MHz, CDCl₃) (δ, ppm): 6.08, 5.53 (d, 12H, CH₂═CCH₃(CO)), 3.20-3.70 (m, 36H, OCH₂), 3.11 (m, 4H, CHOCH, epoxy), 1.92 (s, 18H, CH₂═CCH₃), 0.95-1.50, 2.00-2.20 (m, 54H, CH₂CH₂O, SiCH₂CH₂CHCH₂CH₂CHOCHCH₂, epoxy), 0.49-0.52 (m, 16H, (CH₃)₂SiCH₂), 0.12, 0.10 (d, 48H, (CH₃)₂SiCH₂). ¹³C NMR (100 MHz, CDCl₃) (δ, ppm): 166.81 (C═O), 136.63 (CH₃C═CH₂), 125.10 (CH₃C═CH₂), 75.77-69.76 (OCH₂), 53.10-51.80 (CHOCH α, CHOCH β, epoxy), 35.31-25.34 (CHCH₂CH₂CHOCHCH₂ α, CHCH₂CH₂CHOCHCH₂ β, epoxy), 23.97, 23.52 (SiCH₂CH₂CH α, SiCH₂CH₂CH β), 23.10 (OCH₂CH₂CH₂O), 18.22 (CH₃C═CH₂), 17.31-16.20 (CH₂CH₂CH₂O), 14.49, 14.39 (SiCH₂CH₂ α, SiCH₂CH₂ β, epoxy), 13.65 (Si(CH₃)₂CH₂), −0.42 (Si(CH₃)₂). ²⁹Si NMR (79 MHz, CDCl₃) (δ, ppm): 13.2 (Si(CH₃)₂), −108.8 (SiO₄). FTIR (cm⁻¹): 1719 (C═O), 1638 (C═C), 1254 (Si(CH₃)₂), 1094 (Si—O—Si), 910 (C—O—C, epoxy).

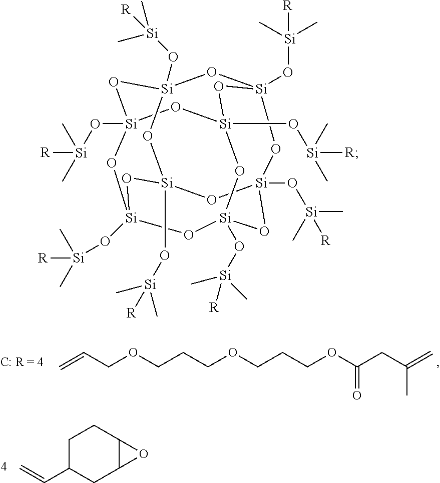

Compound C: ¹H NMR (400 MHz, CDCl₃) (δ, ppm): 6.07, 5.52 (d, 8H, CH₂═CCH₃(CO)), 3.20-3.70 (m, 24H, OCH₂), 3.10 (m, 8H, CHOCH, epoxy), 1.92 (s, 12H, CH₂═CCH₃), 0.95-1.50, 2.00-2.20 (m, 60H, CH₂CH₂O, SiCH₂CH₂CHCH₂CH₂CHOCHCH₂, epoxy), 0.49-0.52 (m, 16H, (CH₃)₂SiCH₂), 0.12, 0.10 (d, 48H, (CH₃)₂SiCH₂). ¹³C NMR (100 MHz, CDCl₃) (δ, ppm): 166.81 (C═O), 136.63 (CH₃C═CH₂), 125.10 (CH₃C═CH₂), 75.75-69.76 (OCH₂), 53.10-51.80 (CHOCH α, CHOCH β, epoxy), 35.31-25.34 (CHCH₂CH₂CHOCHCH₂ α, CHCH₂CH₂CHOCHCH₂ β, epoxy), 23.97, 23.52 (SiCH₂CH₂CH α, SiCH₂CH₂CH β), 23.10 (OCH₂CH₂CH₂O), 18.22 (CH₃C═CH₂), 17.31-16.20 (CH₂CH₂CH₂O), 14.49, 14.39 (SiCH₂CH₂ α, SiCH₂CH₂ β, epoxy), 13.65 (Si(CH₃)₂CH₂), −0.42 (Si(CH₃)₂). ²⁹Si NMR (79 MHz, CDCl₃) (δ, ppm): 13.2 (Si(CH₃)₂), −108.8 (SiO₄). FTIR (cm⁻¹): 1718 (C═O), 1638 (C═C), 1254 (Si(CH₃)₂), 1089 (Si—O—Si), 910 (C—O—C, epoxy).

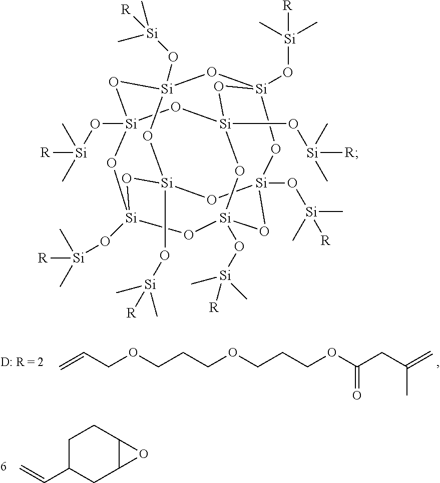

Compound D: ¹H NMR (400 MHz, CDCl₃) (δ, ppm): 6.07, 5.52 (d, 4H, CH₂═CCH₃(CO)), 3.20-3.70 (m, 24H, OCH₂), 3.10 (m, 12H, CHOCH, epoxy), 1.92 (s, 6H, CH₂═CCH₃), 0.95-1.50, 2.00-2.20 (m, 66H, CH₂CH₂O, SiCH₂CH₂CHCH₂CH₂CHOCHCH₂, epoxy), 0.49-0.52 (m, 16H, (CH₃)₂SiCH₂), 0.13, 0.11 (d, 48H, (CH₃)₂SiCH₂). ¹³C NMR (100 MHz, CDCl₃) (δ, ppm): 166.81 (C═O), 136.63 (CH₃C═CH₂), 125.10 (CH₃C═CH₂), 75.70-70.10 (OCH₂), 53.10-51.80 (CHOCH α, CHOCH β, epoxy), 35.31-25.34 (CHCH₂CH₂CHOCHCH₂ α, CHCH₂CH₂CHOCHCH₂ β, epoxy), 23.97, 23.52 (SiCH₂CH₂CH α, SiCH₂CH₂CH β), 23.10 (OCH₂CH₂CH₂O), 18.22 (CH₃C═CH₂), 17.31-16.20 (CH₂CH₂CH₂O), 14.49, 14.39 (SiCH₂CH₂ α, SiCH₂CH₂ β, epoxy), 13.65 (Si(CH₃)₂CH₂), −0.42 (Si(CH₃)₂). ²⁹Si NMR (79 MHz, CDCl₃) (δ, ppm): 13.0 (Si(CH₃)₂), −108.9 (SiO₄). FTIR (cm⁻¹): 1718 (C═O), 1638 (C═C), 1253 (Si(CH₃)₂), 1087 (Si—O—Si), 910 (C—O—C, epoxy).

### Example 4

### Pt-Catalyzed reaction of Propargyl Methacrylate with Octakis(dimethylsiloxy)silsesquioxane

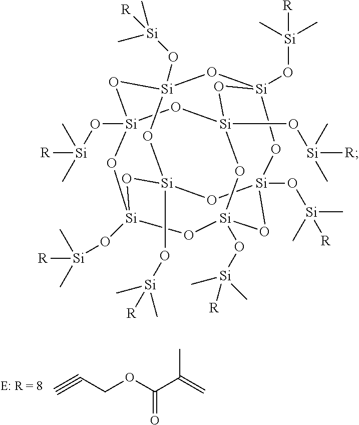

Reaction E (FIG. 3) was carried out with similar procedures to that of reaction A in which the di(propylene glycol)allyl ether methacrylate was replaced with propargyl methacrylate. The reaction mixture was allowed to stir at 50° C. for 24 h. As before, reaction completion was monitored by the disappearance of the Si—H absorption peak at 2145 cm⁻¹ using FTIR. Small amount of excess reactant and solvent were removed effectively by vacuum evaporation technique using a high vacuum pump. A viscous liquid with a product yield of 91% was obtained. ¹H NMR (400 MHz, CDCl₃) (δ, ppm): 6.16-5.59 (m, 32H, CH═CHSi β-trans, CH₂═CCH₃(CO), CH₂═CSi α, CH═CHSi β-trans), 4.81-4.69 (m, 16H, OCH₂C(sp²)), 1.98 (s, 24.0H, CH₂═CCH₃), δ 0.29-0.25 (m, 48.0H, OSi(CH₃)₂CH). ¹³C NMR (100 MHz, CDCl₃) (δ, ppm): 166.8 (C═O), 144.7 (SiC═CH₂ α), 141.1 (CH═CHSi β-trans), 136.2 (CH₂═CCH₃(CO)), 130.0 (CH═CHSi β-trans), 127.0 (SiC═CH₂ α), 125.6 (CH₂═CCH₃(CO)), 67.3 (OCH₂CH═CH β-trans), 66.3 (OCH₂CSi α), 18.3 (CH₂═CCH₃(CO)), −0.14 (OSi(CH₃)₂CH). ²⁹Si NMR (79 MHz, CDCl₃) (δ, ppm): 1.39, 1.04 (Si(CH₃)₂ β-trans α), −109.4 (SiO₄). FTIR (cm⁻¹): 1722 (C═O), 1638 (C═C), 1256 (Si(CH₃)₂), 1092 (Si—O—Si).

### Example 5

### Pt-Catalyzed reaction of Propargyl Methacrylate and 4-Vinyl-cyclohexene Epoxide with Octakis(dimethylsiloxy)silsesquioxane

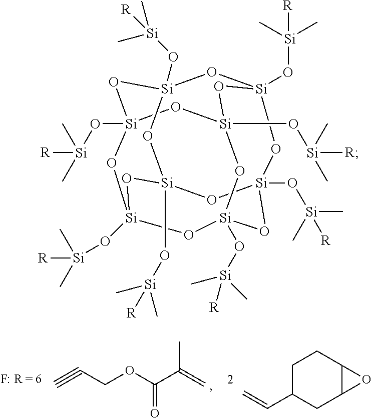

A procedure identical to that of reaction E was followed in which the di(propylene glycol)allyl ether methacrylate was replaced with propargyl methacrylate in step 2 and the mixture was allowed to stir at 50° C. for 24 h. Reaction completion was monitored by the disappearance of Si—H absorption peak at 2145 cm⁻¹ using FTIR. Small amount of excess reactant and solvent were removed effectively by vacuum evaporation technique using a high vacuum pump. Viscous liquid with a product yield of 87% was obtained. The above synthetic procedures were repeated for the synthesis of compounds G and H. Compound F: ¹H NMR (400 MHz, CDCl₃) (δ, ppm): 6.17-5.57 (m, 24H, CH═CHSi β-trans, CH₂═CCH₃(CO), CH₂═CSi α, CH═CHSi β-trans), 4.79-4.66 (m, 12H, OCH₂C(sp²)), 3.14 (m, 4H, CHOCH, epoxy), 1.96 (s, 18H, CH₂═CCH₃), 0.80-1.85, 2.00-2.20 (m, 22H, CH₂CH₂O, SiCH₂CH₂CHCH₂CH₂CHOCHCH₂, epoxy), 0.50-0.55 (m, 4H, (CH₃)₂SiCH₂), 0.27-0.11 (m, 48.0H, OSi(CH₃)₂, OSi(CH₃)₂, epoxy). ¹³C NMR (100 MHz, CDCl₃) (δ, ppm): 166.8 (C═O), 144.8 (SiC═CH₂ α), 141.1 (CH═CHSi β-trans), 136.2 (CH₂═CCH₃(CO)), 130.1 (CH═CHSi β-trans), 126.9 (SiC═CH₂ α), 125.6 (CH₂═CCH₃(CO)), 67.3 (OCH₂CH═CH β-trans), 66.3 (OCH₂CSi α), 53.1-51.8 (CHOCH α, CHOCH β, epoxy), 35.3-25.3 (CHCH₂CH₂CHOCHCH₂ α, CHCH₂CH₂CHOCHCH₂ β, epoxy), 24.0, 23.5 (SiCHCH₃CH α, SiCH₂CH₂CH β, epoxy), 18.3 (CH₂═CCH₃(CO)), 14.5, 14.4 (SiCHCH₃ α, SiCH₂CH₂ β, epoxy), −0.05, −0.47 (Si(CH₃)₂). ²⁹Si NMR (79 MHz, CDCl₃) (δ, ppm): 13.5 (Si(CH₃)₂, epoxy), 1.45, 0.33 (Si(CH₃)₂ β-trans, α), −108.8 (SiO₄). FTIR (cm⁻¹): 1722 (C═O), 1638 (C═C), 1255 (Si(CH₃)₂), 1089 (Si—O—Si).

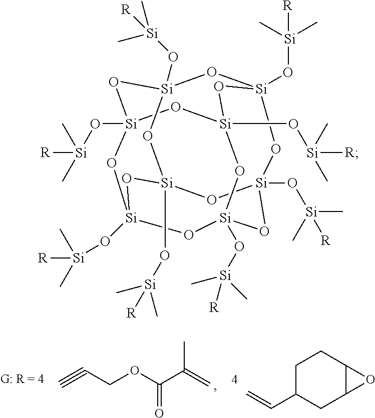

Compound G: ¹H NMR (400 MHz, CDCl₃) (δ, ppm): 6.14-5.58 (m, 16H, CH═CHSi β-trans, CH₂═CCH₃(CO), CH₂═CSi α, CH═CHSi β-trans), 4.79-4.68 (m, 8H, OCH₂C(sp²)), 3.12 (m, 8H, CHOCH, epoxy), 1.96 (s, 12H, CH₂═CCH₃), 0.80-1.90, 2.00-2.20 (m, 44H, CH₂CH₂O, SiCH₂CH₂CHCH₂CH₂CHOCHCH₂, epoxy), 0.48-0.6 (m, 8H, (CH₃)₂SiCH₂), 0.28-0.11 (m, 48.0H, OSi(CH₃)₂, OSi(CH₃)₂, epoxy). ¹³C NMR (100 MHz, CDCl₃) (δ, ppm): 166.7 (C═O), 144.9 (SiC═CH₂ α), 141.2 (CH═CHSi β-trans), 136.3 (CH₂═CCH₃(CO)), 130.2 (CH═CHSi β-trans), 126.9 (SiC═CH₂ α), 125.4 (CH₂═CCH₃(CO)), 67.2 (OCH₂CH═CH β-trans), 66.3 (OCH₂CSi α), 52.9-51.7 (CHOCH α, CHOCH β, epoxy), 35.3-25.3 (CHCH₂CH₂CHOCHCH₂ α, CHCH₂CH₂CHOCHCH₂ β, epoxy), 24.0, 23.5 (SiCHCH₃CH α, SiCH₂CH₂CH β, epoxy), 18.2 (CH₂═CCH₃(CO)), 14.5, 14.4 (SiCHCH₃ α, SiCH₂CH₂ β, epoxy), −0.09, −0.51 (Si(CH₃)₂). ²⁹Si NMR (79 MHz, CDCl₃) (δ, ppm): 13.15 (Si(CH₃)₂, epoxy), 1.05, 0.64 (Si(CH₃)₂ β-trans, α), −109.0 (SiO₄). FTIR (cm⁻¹): 1722 (C═O), 1638 (C═C), 1254 (Si(CH₃)₂), 1089 (Si—O—Si).

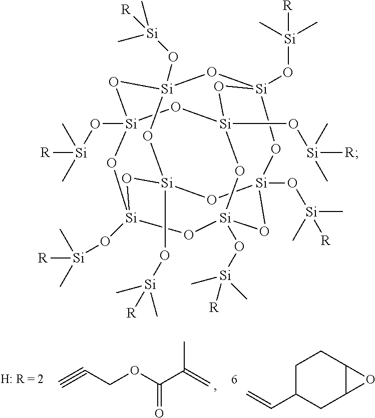

Compound H: ¹H NMR (400 MHz, CDCl₃) (δ, ppm): 6.15-5.59 (m, 8H, CH═CHSi β-trans, CH₂═CCH₃(CO), CH₂═CSi α, CH═CHSi β-trans), 4.81-4.69 (m, 4H, OCH₂C(sp²)), 3.16 (m, 12H, CHOCH, epoxy), 1.97 (s, 6H, CH₂═CCH₃), 0.80-1.90, 2.03-2.19 (m, 66H, CH₂CH₂O, SiCH₂CH₂CHCH₂CH₂CHOCHCH₂, epoxy), 0.50-0.60 (m, 12H, (CH₃)₂SiCH₂), 0.28-0.13 (m, 48.0H, OSi(CH₃)₂, OSi(CH₃)₂, epoxy). ¹³C NMR (100 MHz, CDCl₃) (δ, ppm): 166.7 (C═O), 144.9 (SiC═CH₂ α), 141.1 (CH═CHSi β-trans), 136.2 (CH₂═CCH₃(CO)), 130.2 (CH═CHSi β-trans), 126.9 (SiC═CH₂ α), 125.9 (CH₂═CCH₃(CO)), 67.2 (OCH₂CH═CH α-trans), 66.3 (OCH₂CSi α), 52.9-51.7 (CHOCH α, CHOCH β, epoxy), 35.3-25.3 (CHCH₂CH₂CHOCHCH₂ α, CHCH₂CH₂CHOCHCH₂ β, epoxy), 24.0, 23.5 (SiCHCH₃CH α, SiCH₂CH₂CH β, epoxy), 18.2 (CH₂═CCH₃(CO)), 14.5, 14.4 (SiCHCH₃ α, SiCH₂CH₂ β, epoxy), −0.10, −0.50 (Si(CH₃)₂). ²⁹Si NMR (79 MHz, CDCl₃) (δ, ppm): 13.15 (Si(CH₃)₂, epoxy), −1.20, −2.21 (Si(CH₃)₂ β-trans, α), −108.8 (SiO₄). FTIR (cm⁻¹): 1722 (C═O), 1638 (C═C), 1253 (Si(CH₃)₂), 1093 (Si—O—Si).

### Example 6

### Formulation of Light-activated Experimental Nanocomposites

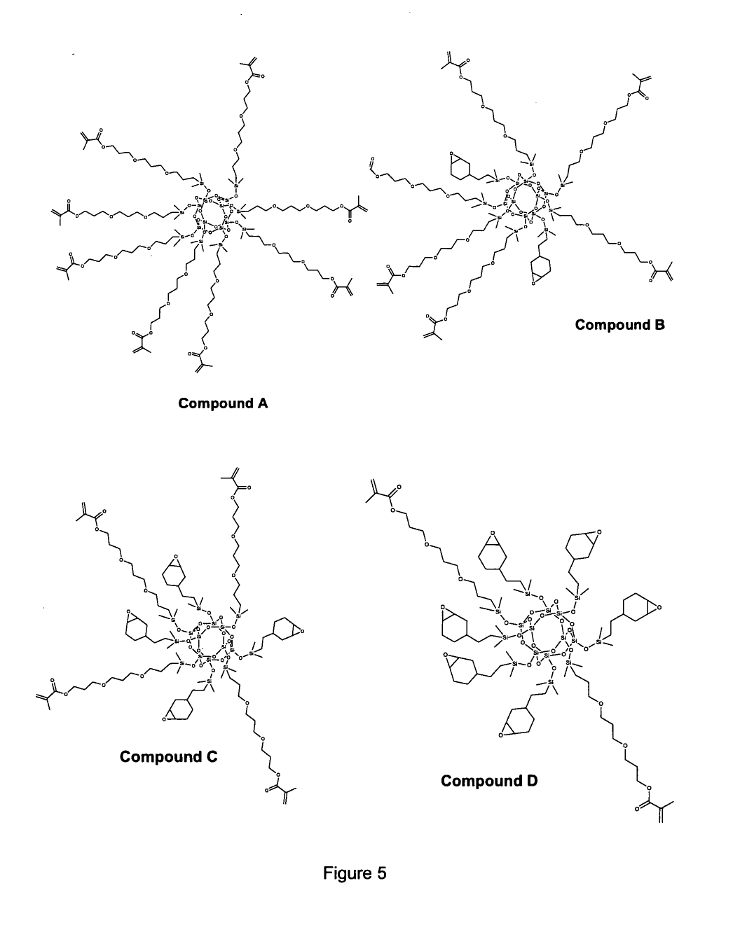

The synthesized SSQ compounds were blended with control monomers in 5, 10, 20 and 50 wt % of SSQ resins ratios respectively. The control material in this study was made by mixing Bis-GMA [bisphenol A glycerolate (1 glycerol/phenol) dimethacrylate] and TEGDMA [tri(ethylene glycol) dimethacrylate] in a ratio of 1:1 for optimum properties. Preparation of light-activated experimental SSQ resins involved the addition of 1 mol % of both visible light initiators (camphorquinone, CQ) and activators (2-(dimethylamino) ethyl methacrylate, DMAEMA) to the monomers followed by addition of 63 wt % silanated silica fillers (G018-0166, SCHOTT Electronic Packaging GmbH). The fillers with an average particles size of 1.5 microns were silanated with 3.2% of silane coupling agent (γ-methacryloxypropyltrimethoxysilane). The various composite formulations were then mixed homogeneously by hand spatula in light-tight containers with care taken to ensure that no air bubbles were trapped upon mixing. Table 1 shows the composition of the respective materials. FIG. 5 illustrates the inventive compounds used in these examples.

TABLE 1

| | | Shade | |
|---|---|---|---|
| | | & Lot | |
| Material | Manufacturer | number | Composition* |
| Filtek ™ | 3M-ESPE, St. | A2B | Resins: Bis-GMA, UDMA, Bis-EMA (6) and |
| Supreme | Paul, MN 55144 | 2AE | TEGDMA (small amounts) |
| [FS] | | | Fillers: Non-agglomerated/non-aggregated |
| | | | nanosilica (20 nm), agglomerated |
| | | | zirconia/silica nanocluster (0.6-1.4 microns), |
| | | | consisting of agglomerates of primary |
| | | | zirconia/silica particles with size of 5-20 nm |
| | | | fillers |
| | | | Filler by weight: 78.5% |
| Filtek ™ | 3M-ESPE, St. | A2D | Resins: Bis-GMA and TEGDMA |
| A110 | Paul, MN 55144 | 2BA | Fillers: Silica (0.04 microns) |
| | | | Filler by weight: 56% |
| Filtek ™ | 3M-ESPE, St. | A2 | Resins: Bis-GMA, TEGDMA and Proprietary |
| Flow | Paul, MN 55144 | 3CG | dimethacrylate polymer |
| [FF] | | | Fillers: Zirconia/silica (1.5 microns) |
| | | | Filler by weight: 68% |
| S1 | Self-synthesized | | Resins: Bis-GMA, TEGDMA, 10% compound C |
| | | | Fillers: Silica (1.5 microns) |
| | | | Filler by weight: 63% |
| S2 | Self-synthesized | | Resins: Bis-GMA, TEGDMA, 20% compound C |
| | | | Fillers: Silica (1.5 microns) |
| | | | Filler by weight: 63% |
| S3 | Self-synthesized | | Resins: Bis-GMA, TEGDMA, 20% compound B |
| | | | Fillers: Silica (1.5 microns) |
| | | | Filler by weight: 63% |
| S4 | Self-synthesized | | Resins: Bis-GMA, TEGDMA, 20% compound D |
| | | | Fillers: Silica (1.5 microns) |
| | | | Filler by weight: 63% |

*All compounds used are commercial products except for compounds B, C and D which have been prepared according to the process of the present invention.

### Physical/Mechanical Characterizations

### Polymerization Shrinkage

Post-gel polymerization shrinkage was determined using the experimental set-up used by Yap et al.,^{28,29} with minor modifications. A white Teflon mold of inner length 7.0 mm, width 4.0 mm and height 2.0 mm was used to confine the synthesized materials with the exception of a window for the strain gauge leads. Foil electrical resistance strain gauges (Foil Strain Gauge, RS Components Ltd, Singapore) of 2 mm in length, an electrical resistance 120Ω and gauge factor 2.00 were positioned onto the flat surfaces of the Teflon mold. With the strain gauges in place, the synthesized materials were placed into the cavity of the Teflon mold. Care was taken to ensure complete filling of the cavity and excess material was extruded using pressure applied through a glass slide. The leads from the strain gauge were connected to a strain-monitoring device (Strain Gauge Recorder, Cole Parmer Instruments, IL 60061) initially balanced at zero. The strain-monitoring device consisted of a chart recorder which functions by rationing sense voltage to signal voltage and converting it to analog output. Dimensional changes are thus effectively transferred to the gauges and measured in terms of resistance.

The test samples were then light polymerized at 500 mW/cm² for 40 seconds. A total of three specimens were made for each material. Dimensional change during and post light polymerization was monitored in air at room temperature (25±1° C.). Post light polymerization shrinkage measurements were taken at 0 (immediately after light polymerization), 1, 10, 30 and 60 minutes after removal of the curing light. Percentage linear shrinkage was derived from the following equation (1):

$$Percentage linear shrinkage == \frac{Δ L}{L} \times 100 = \frac{Δ R / R}{K} \times 100 \qquad ( 1 )$$

where ΔL=Change in length, L=Original length, ΔR=Change of resistance, R=Original resistance and K=Gauge factor (i.e. 2).

### Indentation Hardness and Elastic Modulus

Both indentation hardness and elastic modulus were determined using a depth-sensing microindentation technique employed by Yap et al.³⁰ Synthesized materials were placed into the square recess (3 mm length, 3 mm width and 2 mm height) of customized acrylic molds with excess materials extruded using pressure applied through a glass slide. The synthesized materials were then light polymerized at 500 mW/cm² for 40 seconds. Both hardness and modulus were determined at 0 (immediately after cure), 1 and 7 days with specimens conditioned in distilled water at 37° C. A total of seven specimens were made for each synthesized material.

Depth-sensing microindentation for both hardness and modulus was carried out using a custom designed indentation head unit mounted to an Instron Micro-Test system (Model 5848, Instron Corporation, Canton, USA). The specimens were indented with a Vickers indenter at a rate of 0.0005 mm/s until a maximum load of 10N was attained. The peak load was then held for 10 s and unloaded fully at a rate of 0.0002 mm/s. Indentation loads (P) and the corresponding indenter displacements (h) were recorded continuously during the loading-unloading cycle. Hardness (H) data were obtained by dividing the peak load (Pₘₐₓ) over the maximum projected contact area (Aₘₐₓ) (equation 2) while elastic modulus (Eᵢₙ) is calculated by the unloading contact stiffness analysis (S) according to Oliver and Pharr (equation 3).³¹

$$\begin{matrix}H = \frac{{P}_{max}}{{A}_{max}} & ( 2 ) \\ {E}_{in} = \frac{1 - {υ}_{in}^{2}}{( 1.142 \frac{\sqrt{{A}_{max}}}{S} - \frac{1 - {υ}_{o}^{2}}{{E}_{o}} )} & ( 3 ) \\ where {A}_{max} = 24.5 {( {h}_{max} - \frac{3 {P}_{max}}{4 S} )}^{2} & \end{matrix}$$

Eₒ=Elastic modulus of diamond Vickers indenter, 1141 GPa
υₒ=Poisson's ratio of the indenter, 0.07
υᵢₙ=Poisson's ratio of the test material, 0.24
hₘₐₓ=Indentation depth at maximum load

### Degree of Conversion

The degree of conversion was determined using a TA instruments DSC Q100 equipped with a PCA (Photocalorimetric Accessory) unit. Specimens (n=3) of approximately (10±2) mg were weighed and placed in a hermetic DSC aluminum pan. Samples were then exposed to visible light (mercury lamp) for 40 seconds at 500 mW/cm² under N₂ at 37° C. The extent of polymerization was then derived from the heat of photopolymerization (ΔH) calculated from the area under the peak of the differential temperature curve using theoretical reaction enthalpy of 13.1 kcal/mol for the methacrylate double bonds³² as the standard value for heat of polymerization in this study. Degree of conversion was then derived from the following equation (3):

$$Degree of conversion = \frac{Δ {H}_{Experimental}}{Δ {H}_{Theoretical}} \times 100 \qquad ( 3 )$$

### Depth of Cure

The depth of cure testing methodology was carried out by means of a scraping technique based on ISO 4049 (2000). The composite was placed in black teflon molds with square cavities 6.7 mm deep and 4 mm wide/long and confined between two opposing acetate strips (Hawe-Neos Dental, Bioggio, Switzerland). A white delrin base was used beneath the molds. A glass slide (1 mm thick) was then placed on the molds and excess material was extruded by pressure application. The composite was then irradiated from the top through the glass slide and acetate strip at 500 mW/cm² for 40 seconds with the VIP light curing unit. Immediately after light polymerization, the acetate strips were removed followed by the specimens in their molds. Uncured materials were then removed with a plastic spatula. Height of the cured material was measured with a digimatic caliper (Mitutoyo Corporation, Japan). Depth of cure was tabulated as 50 percent of the remaining length. Five specimens were prepared for each composite

### Water Sorption

Water sorption test for all composites were determined using technique specified in ISO 4049 (2000). The composite was placed in white teflon molds of 10 mm in diameter and 1 mm in thickness and confined between two opposing acetate strips (Hawe-Neos Dental, Bioggio, Switzerland). A white delrin base was used beneath the molds. A glass slide (1 mm thick) was then placed on the molds and excess material was extruded by pressure application. The composite was then irradiated from the top through the glass slide and acetate strip at 500 mW/cm² for 40 seconds with the VIP light curing unit. Immediately after light polymerization, the acetate strips were removed followed by the specimens in their molds. On removal, specimens were placed in a desiccator containing freshly dried silica gel maintained at (37±1)° C. Specimens were then removed after 24 hours and stored in a second desiccator maintained at (23±1)° C. for 2 hours and weighed to an accuracy of 0.1 mg. This cycle were repeated until a constant mass (m₁) was obtained. After the final conditioning, both diameter and thickness of each specimen were determined using a digimatic caliper (Mitutoyo Corporation, Japan) and specimen volume (V) were calculated in mm³. All specimens were immersed in 20 ml of distilled water at (37±1)° C. for 7 days then removed, blotted dry and weighed (m₂). After this weighing, specimens were reconditioned to constant mass (m₃) in the dessicator using the above described cycle. Five specimens were prepared for each material. Water sorption, Wsp, in micrograms per cubic millimeter was calculated using the following equation (4):

$$Wsp = \frac{{m}_{2} - {m}_{3}}{V} \qquad ( 4 )$$

### Results of the Experiments:

### Polymerization Shrinkage

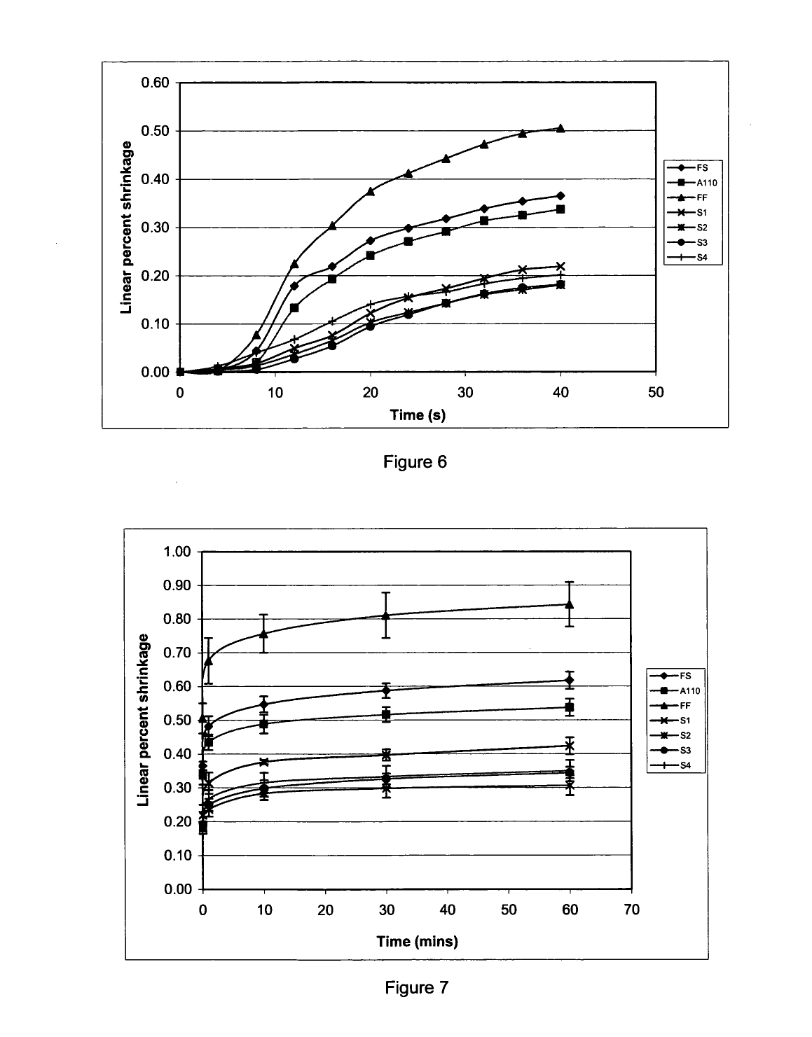

The mean linear percent shrinkage of the various dental composites evaluated during light polymerization is shown in FIG. 6. The mean linear percent shrinkage at the various post light polymerization time intervals is shown in FIG. 7. For all materials, the rate of shrinkage was greatest during light polymerization reaction and continued after removal of the light source (FIG. 6). The shrinkage strain that occurs prior to gelation does not contribute to stress as only viscous but unrestricted flow was involved. Flow was defined as the amount by which the shrinkage stresses exceed the elastic limit and was thought to be the ability of molecules within the forming polymer to slip into new positions before being constrained by cross-linking. In light-activated composites, the fast reaction rate practically eliminates the time allowed for viscous flow, and that the polymer matrix becomes rigid within seconds after a relatively low degree of conversion. The polymerization of the resin matrix involves the gelation process whereby the restorative material is transformed from a viscous-plastic into a rigid-elastic phase. When flow ceases after gelation and can no longer compensate for shrinkage stresses, post-gel polymerization shrinkage develops. This polymerization process is accompanied by a rapid increase in elastic modulus which induces stress within the polymer and distributes it to the boundary layers. This post-gel shrinkage influences the strength of the bond between composite resins and tooth structure which may lead to bond failure arising from defects in the composite-tooth bond.

At all time intervals, the post-gel shrinkage associated with the flowable composites, FF was found to be significantly greater than all the other dental composites evaluated. The low viscosity resin composites were developed for ease of placement and handling. As the volume fraction of polymer matrix in the composite and the type of polymers used are primary material factors influencing polymerization shrinkage the high shrinkage obtained with FF may be attributed to the proprietary dimethacrylate polymer used.

Post-gel shrinkage associated with experimental nanocomposites was found to be significantly lower than A110 with the exception of S1. Although shrinkage value associated with S1 was low, no significant difference was observed between that of S1 and A110. The low shrinkage property obtained with all experimental nanocomposites may be attributed to the presence of the synthesized SSQ compounds. Post-gel shrinkage decrease with increase in SSQ compounds added. This accounted for the differences in shrinkage values between that of S1 and S2. S1 with only 10 wt % compound C incorporated have more Bis-GMA/TEGDMA monomers in the composite system when compared to S2. The low molecular weight TEGDMA, being more flexible and having more double bonds per unit of weight, accounted for the relatively higher polymerization shrinkage of S1.

When comparing across the different nanocomposites S2, S3 and S4, post-gel shrinkage generally decrease with decrease in the number of methacrylate chains attached to SSQ with the exception of S4. S4 with 2 long chains of methacrylate groups and 6 short chains of epoxy groups attached to the SSQ core was found to be more accessible and have better interaction with the control monomers. This accounted for the slight difference in shrinkage values.

### Indentation Modulus and Hardness

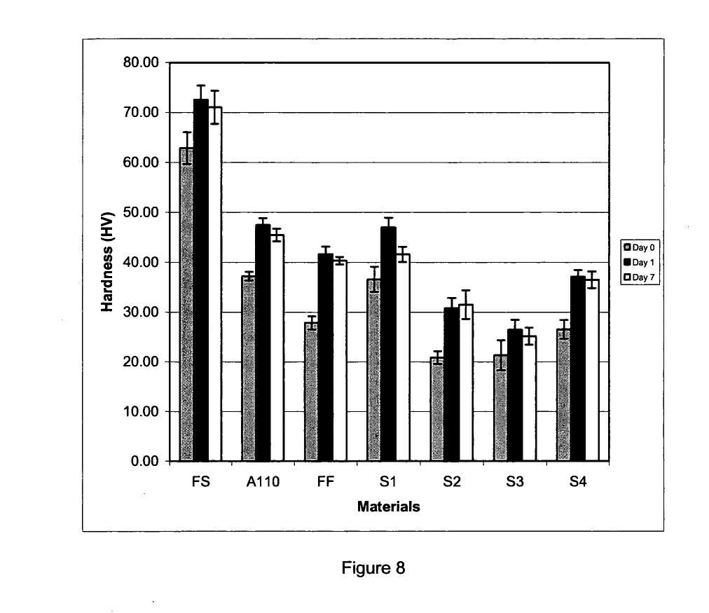

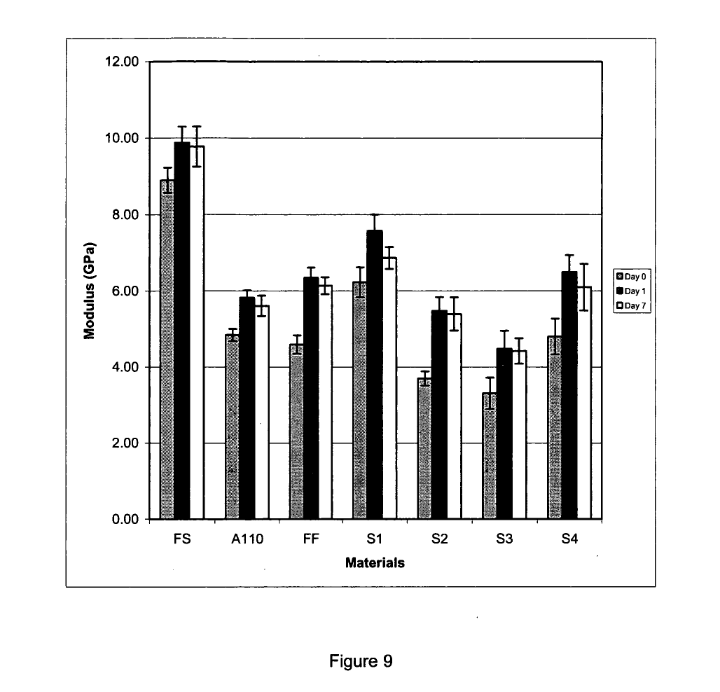

The mean hardness and modulus of FS, A110, FF and all experimental nanocomposites (S1-S4) are shown in FIGS. 8 and 9 respectively. The understanding of the mechanical properties of dental composites is important for the development of “tooth-like” restorative materials. An ideal restorative material is one that has mechanical properties comparable to those of enamel and dentin. Both hardness and elastic modulus are amongst the several mechanical properties evaluated in this study to determine the resistance to occlusal forces. Hardness refers to the resistance of a material to indentation or penetration. It has also been related to strength, proportional limit and ductility of materials and has been used to predict the wear resistance of a material and its ability to abrade or be abraded by opposing tooth structure and materials. On the other hand, elastic modulus refers to the relative stiffness of a material. Together with adhesive properties, elastic modulus plays an important role in preventing microleakage, secondary caries and/or filling dislodgement.

From results obtained in FIGS. 8 and 9, indentation hardness and modulus associated with FS at day 0 (immediately after cure), 1 and 7 was found to be significantly higher than all materials evaluated. This may be attributed to the amount of fillers present in the composite system. FS which consists of both nanosilica and zirconia/silica nanocluster has fillers loading of 78.5 wt %. The combination of nanosized particles and nanocluster reduces interstitial spacing of the fillers particles and thus results in higher fillers loading and better mechanical properties.

At all time intervals, no significant difference in hardness was observed between that of S1, A110 and FF in general while the elastic modulus of S1 was found to be significantly higher than A110 and FF. The high hardness and modulus values of S1 can be attributed to the addition of 10 wt % compound C. The addition of SSQ compounds often results in dramatic improvements in polymer properties due to their ability to bridge the gap between inorganic ceramics and organic polymers. Results obtained for FF corroborated well with that of polymerization shrinkage which stated that FF tended to shrink more and was less rigid than conventional hybrid materials due to their increased flow capacity.

### Degree of Conversion

The mean degree of conversion of the various materials evaluated is shown in Table 2. Degree of conversion associated with the commercial dental composites was found to be significantly greater than all experimental nanocomposites except S4. S4 was found to have a degree of conversion higher than A110.

TABLE 2

| Materials | Degree of Conversion |
|---|---|
| FS | 60.61 (1.62) |
| A110 | 47.70 (2.64) |
| FF | 67.67 (2.25) |
| S1 | 39.58 (0.98) |
| S2 | 40.85 (0.58) |
| S3 | 36.61 (1.43) |
| S4 | 54.84 (1.71) |

The degree of conversion in a cross-linked polymeric system plays a potentially significant role in determining the ultimate physical and mechanical properties of the material. Inadequate polymerization results in inferior physico-mechanical properties such as poor resistance to wear, poor color stability, secondary caries and adverse tissue reactions, increased rates of water sorption, solubility and early restoration failure. While, it is desirable for dental composite resins to achieve high levels of conversion, there is usually a significant concentration of unreacted carbon double bonds remaining in the resin when cured. This is due to limitations on the mobility of reactive species imposed by the rapid formation of a cross-linked polymeric network. In addition, high levels of conversion also resulted in higher contraction strain rates during polymerization. This usually results in gaps around the cavity margins, resulting in microleakage, pulpal irritation, thermal sensitivity, recurrent caries and internal stresses.

Various techniques used for the measurement of the degree of conversion include Fourier Transform Infrared Spectroscopy (FTIR), Laser Raman, Micro-Raman Spectroscopy, MIR (multiple internal reflection), NIR (near infrared), ATR and micro-attenuated total reflection infrared spectroscopy (micro-ATR), differential thermal analysis (DTA) and differential scanning calorimetry (DSC). In the following, the DSC technique with a photopolymerization apparatus was chosen for the measurement of the degree of conversion for its simplicity. It is a convenient tool which provides a measure of methacrylate conversion based on the enthalpy of the exothermic polymerization.

From Table 2, degree of conversion associated with FF was found to be significantly greater than all other materials evaluated. This phenomenon can be explained by the flowability of the composites as a more viscous materials tended to restrict the mobility of reactive species and reduce the frequency and probability of random encounters, which led to a decrease in polymerization propagation.

For the degree of conversion of the experimental nanocomposites developed, S4 was found to have a degree of conversion greater than S1, S2 and S3. No significant difference was observed between that of S4, FS and A110. This may be due to the addition of the more flexible and accessible compound D. When the methacrylate groups of compound D start to react with the control on polymerization, they bring with them the attached SSQ cubes. After the first coupling reactions, the SSQ cubes are anchored into the crosslink network. With the restrictions set by the bulky SSQ cubes, molecular structures of the network were set. Further reactions involved only movement of both the small molecules and anchored methacrylate groups. As compound 2 has only 2 long chain methacrylate groups attached, more interstitial spacing were present in the network after the first coupling when compared to S2 and S3. This interstitial spacing may be filled up by the smaller Bis-GMA and TEGDMA molecules on further reactions. Thus, this may account for the high degree of conversion and low shrinkage properties obtained for S4.

In this study, the obtained degree of conversion of the different materials corroborated well with earlier studies where degree of conversion was found to range from 43.5 to 73.8% and was highest for the most diluted resins. However, it must be noted that the degree of conversion does not indicate the degree of polymerization of Bis-GMA or TEGDMA itself but indicates the conversion rate of the aliphatic C═C bond in the methyl methacrylate group into a C—C bond. The 51% of degree of conversion does not mean that that 49% of monomer remains but indicates that 49% of C═C bonds remain. Though degree of conversion is maximized by the inclusion of a high percentage (40-50%) of diluents in the resin, the cure is accompanied by significant polymerization shrinkage (2-14 vol %) for most commercial materials.

### Depth of Cure

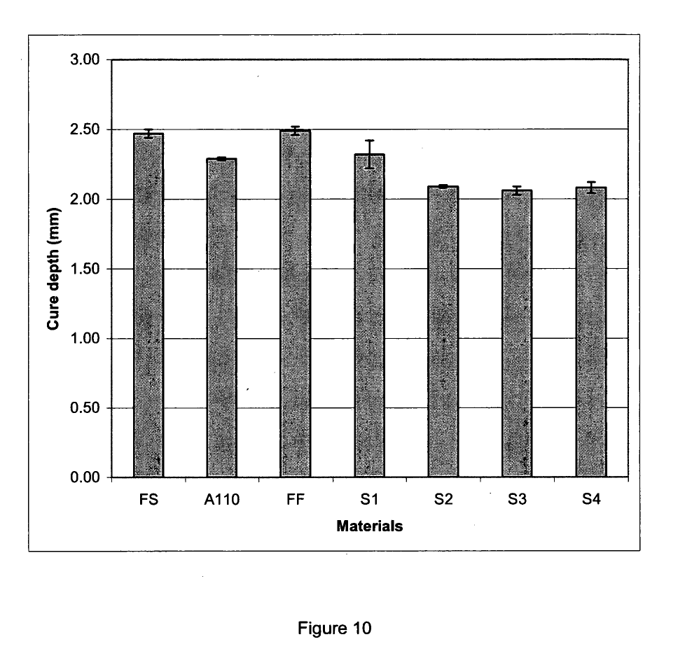

In addition to the polymerization shrinkage, hardness, modulus and degree of conversion, the depth of cure is another important property to be determined. FIG. 10 show the mean depth of cure of FS, A110, FF and S1-S4. As light pass through the bulk of the composite to initiate polymerization, it is absorbed and scattered. These factors result in an attenuation of light intensity as it passes through the restoration bulk. The result of this attenuation is that cure on the surface is much greater than it is within the depths of the material. At greater depths, part of the light required for further polymerization is absorbed by the already polymerized layers of composite resins. Thus, depth of cure can be defined as the extent of quality resin cure deep down from the surface of composite restoratives and is affected by filler size, light source intensity, duration of exposure and resin shade. As aforementioned, the presence of inadequate polymerization throughout the depth of restoration can lead to undesirable effects such as gap formation, marginal leakage, recurrent caries, adverse pulpal effects and ultimate failure of restoration.

Various techniques used for the determination of depth of cure of composites include an optical microscope where changes in the translucency of light-cured composite resins are detected, the ISO 4049 scraping technique, knoop hardness testing, the digital penetrometer test method, dye uptake, tactile tests and nuclear magnetic resonance microimaging. In this study, the ISO scraping technique was chosen for the determination of depth of cure due to its relative simplicity and minimal instrumentation requirement. It is a simple method that enables relative comparison of the curing depth and is suitable for clinicians to perform.

### Water Sorption

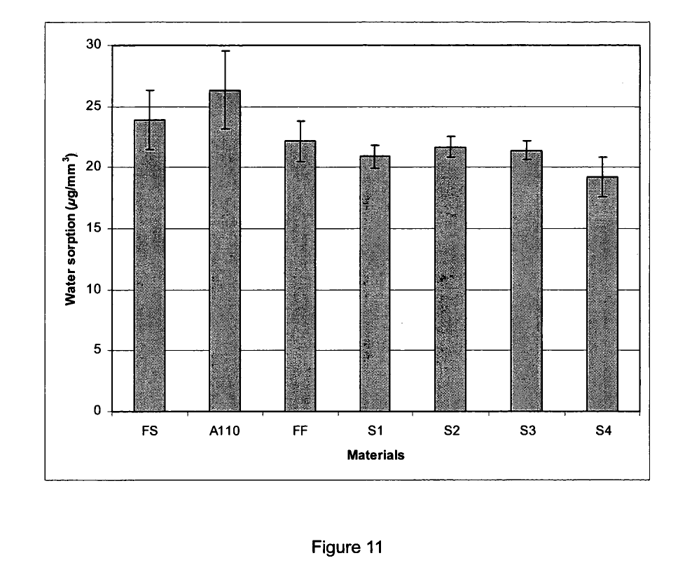

Results of the mean water sorption evaluated for the different materials are shown in FIG. 11. The water sorption by composite materials in an aqueous environment is defined as a diffusion-controlled process with water uptake occurring mainly in the resin matrix. While water sorption by polymer network contributes to stress reduction, its effect is minimized as water uptake by composite resins takes place at a much slower rate, requiring hours to reach saturation. In addition, the water sorbed by the polymer matrix could cause filler-matrix debonding or even hydrolytic degradation of the fillers, and may affect composite materials by reducing their mechanical properties and wear resistance.

From results obtained in FIG. 10, all materials evaluated met the ISO water sorption requirement of less than 40 μg/mm³. The water sorption associated with commercial composites, FF, FS and A110 was found to be greater than all experimental nanocomposites S1-S4. The higher water sorption values obtained with FF, FS and A110 when compared to S1-S4 could be attributed to the different chemical structures and fillers loading. Studies have shown that water sorption is highly dependent upon the chemistry of the monomers. The water sorption of various monomers was found to be as follows: TEGDMA>Bis-GMA>UDMA>Bis-EMA. The presence of the hydrophilic ether linkages in TEGDMA, hydroxyl groups of Bis-GMA and urethane linkages in UDMA accounted for the differences in water sorption. Thus, the existence of both hydrophilic TEGDMA/Bis-GMA and low fillers loading (56 wt %) of A110 accounted for the high water sorption obtained.

### CONCLUSIONS

Within the limitations of this in-vitro study, post-gel shrinkage associated with S1-S4 was found to be significantly lower than all commercial composites evaluated. No significant difference in hardness was observed for S1, A110 and FF. Modulus associated with S1 and S4 was found to be higher if not equal to A110 and FF. The degree of conversion of S4 was also found to be higher than A110. All materials evaluated for depth of cure and water sorption met the ISO requirements. Thus, the development of experimental nanocomposites, for example S4, does show potential for use as low-shrinking dental restoratives with physico-mechanical properties comparable to some commercial dental composites. It was believed that with further modifications and optimization on the filler particles used, polymerization shrinkage and physico-mechanical properties may be further enhanced.

### REFERENCES

The following references are cited in the present application:
1. Leinfelder K F. New developments in resin restorative systems. J Am Dent Assoc 1997; 128:573-581.
2. Soh M S, Yap A U, Siow K S. Post-gel shrinkage with different modes of LED and halogen light curing units. Oper Dent 2004; 29:317-324.
3. Labella R, Lambrechts P, Van Meerbeek B, Vanherle G. Polymerization shrinkage and elasticity of flowable composites and filled adhesives. Dent Mater 1999; 15:128-137.
4. Yap A U, Wang H B, Siow K S, Gan L M. Polymerization shrinkage of visible light-cured composites. Oper Dent 2000; 25:98-103.
5. Feilzer A J, Dooren L H, de Gee A J, Davidson C L. Influence of light intensity on polymerization shrinkage and integrity of restoration-cavity interface. Eur J Oral Sci 1995; 103:322-326.
6. Price R B, Rizkalla A S, Hall G C. Effect of stepped light exposure on the volumetric polymerization shrinkage and bulk modulus of dental composites and an unfilled resin. Am J Dent 2000; 13:176-180.
7. Davidson C L, de Gee A J. Relaxation of polymerization shrinkage stresses by flow in dental composites. J Dent Res 1984; 63:146-148.
8. Feilzer A J, de Gee A J, Davidson C L. Setting stress in composite resin in relation to configuration of the restoration. J Dent Res 1987; 66:1636-1639.
9. Eick J D, Welch F H. Polymerization shrinkage of posterior composite resins and its possible influence on postoperative sensitivity. Quintessence Int 1986; 17:103-111.
10. Leevailoj C, Cochran M A, Matis B A, Moore B K, Platt J A. Microleakage of posterior packable resin composites with and without flowable liners. Oper Dent 2001; 26:302-307.
11. Bowen R L. Adhesive bonding of various materials to hard tooth tissues. VI. Forces developing in direct-filling materials during hardening. J Am Dent Assoc 1967; 74:439-445.
12. Sheth J J, Fuller J L, Jensen M E. Cuspal deformation and fracture resistance of teeth with dentin adhesives and composite. J Prosthet Dent 1988; 60:560-569.
13. Stansbury J W. Synthesis and evaluation of new oxaspiro monomers for double ring-opening polymerization. J Dent Res 1992; 7:1480-1412.
14. Miyazaki K, Takata T, Endo T, Inanaga A. Thermal and photo-polymerization of methacrylates containing a Spiro ortho ester moiety and the properties of polymethacrylates. Dent Mater 1994; 13:9-18.
15. Nuyken O, Böhner R, Erdmann C. Oxetane photopolymerization—A system with low volume shrinkage. Macromol Symposia 1996; 107:125-128.
16. Stansbury J W. Cyclopolymerizable monomers for use in dental resin composites. J Dent Res 1992; 71:434-437.
17. Klee J E, Neidhart F, Flammersheim H-J. Monomers for low shrinking composites, 2. Synthesis of branched methacrylates and their application in dental composites. Macromol Chem Phys 1999; 200:517-523.
18. Fong H, Dickens S H, Flaim G M. Evaluation of dental restorative composites containing polyhedral oligomeric silsesquioxane methacrylate. Dent Mater 2004; 21:520-529.
19. Weinmann W, Thalacker C, Guggenberger R. Siloranes in dental composites. Dent Mater 2005; 21:68-74.
20. Choi J, Kim S G, Laine R M. Organic/Inorganic Hybrid Epoxy Nanocomposites from Aminophenylsilsesquioxanes. Macromolecules 2004; 37:99-109.
21. Sellinger A, Laine R M. Silsesquioxanes as synthetic platforms. 3. Photocurable, liquid epoxides as inorganic/organic hybrid precursors. Chem Mater 1996; 8:1592-1593.
22. Sellinger A, Laine R M. Silsesquioxanes as synthetic platforms. Thermally curable and photocurable inorganic/organic hybrids. Macromolecules 1996; 29:2327-2330.
23. Laine R M, Zhang C, Sellinger A, Viculis L. Polyfunctional Cubic Silsesquioxane as Building Blocks for Organic/Inorganic Hybrids. Appl. Organometal. Chem. 1998; 12:715-723.
24. Zhang C, Laine R M. Silsesquioxane-liquid crystalline (LC) composites: Potential precursors to dental material. Polym Prep: Div Polym Chem, Amer Chem Soc 1997; 38:120-121.
25. Li G, Wang L, Ni H, Pittman Jr C U. Polyhedral oligomeric silsesquioxane (POSS) polymers and copolymers: A review. J Inorg and Organomet Polym 2001; 11:123-154.
26. Baney R H, Itoh M, Sakakibara A, Suzuki T. Silsesquioxanes. Chem Rev 1995; 95:1409-1430.
27. Gao F, Tong Y, Schricker S R, Culbertson B M. Evaluation of neat resins based on methacrylates modified with methacryl-POSS, as potential organic-inorganic hybrids for formulating dental restoratives. Polym Adv Technol 2001; 12:355-360.
28. Yap, A. U.; Soh, M. S. Oper Dent 2004, 29, 182-187.
29. Yap, A. U.; Wang, H. B.; Siow, K. S.; Gan, L. M. Oper Dent 2000, 25, 98-103.
30. Yap, A. U.; Wang, X.; Wu, X.; Chung, S. M. Biomaterials 2004, 25, 2179-2185.
31. Oliver, W. C.; Pharr, G. M. J Mater Res 1992, 7, 1564-1568.
32. Anseth, K. S.; Wang, C. N.; Bowman, C. N. Polymer 1997, 35, 3243-3250.

1.-37. (canceled)

38. A nanocomposite material comprising
a) polymerizable monomers, the polymerizable monomers comprising at least one multi functionalized silesquioxane (SSQ) derivative according to formula (I)
[((R¹)ₐ(R²)_{b}(R³)_{c}(R⁴)_{d})((R⁵)ₙSiₙO_{z})] (I) as co-monomer;
wherein
n is an integer from 6 to 14;
z is 1.5n;
R¹, R², R³, and R⁴ are independently selected from the group of polymerizable residues consisting of C₂-C₂₀ alkene, C₂-C₂₀ alkyne, allyl, allyl glycidyl ether, C₂-C₂₀ alkylalkene, alkylalkynes, acrylates, methacrylates, benzoxazines, epoxides and oxetanes;
R⁵ is independently selected from (C(R^{I})(R^{II}))_{q}, ((C(R^{I})(R^{II}))_{q}N(R^{III})), [(CH₂)ₘO]ᵣ and [(Si(R^{IV})₂O)]ₛ, wherein R^{I}, R^{II}, R^{III} and R^{IV} are independently selected from hydrogen, C₁-C₂₀ alkyl, and C₆-C₁₀ aryl; m is an integer from 1 to 10; and q, r and s are independently an integer from 0 to 10;
a, b, c and d are independently an integer from 0 to n, wherein a+b+c+d=n,
with the proviso that in case z is 1.5n then at least one of R¹, R², R³ and R⁴ is not (CH₂)₃OC(O)C(CH₂)(CH₃), (CH₂)₃OC(O)C(CH₂)(H), CH(CH₂), 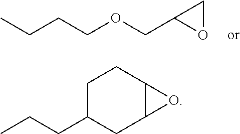
b) a curing system; and
c) at least one filler; and
wherein the SSQ is used in an amount of about 5 wt % to about 40 wt % based on the total amount of the nanocomposite material.

39. The nanocomposite material according to claim 38, wherein the SSQ is used in an amount of about 10 wt % to about 40 wt % based on the total amount of the nanocomposite material.

40. The nanocomposite material according to claim 38, wherein the SSQ is used in an amount of about 10 wt % to about 20 wt % based on the total amount of the nanocomposite material.

41. The nanocomposite material according to claim 38 wherein the C₂-C₂₀ alkene is selected from the group consisting of ethenyl, propenyl, butenyl, 1,4-butadienyl, pentenyl, hexenyl, 4-methylhex-1-enyl and 4-ethyl-2-methylhex-1-enyl.

42. The nanocomposite material according to claim 38, wherein the C₂-C₂₀ alkylalkene is selected from the group consisting of (CH₂)_{w}—CH(CH₂) and (CH₂)_{w}—C(CH₃)(CH₂), wherein w is an integer from 1 to 15.

43. The nanocomposite material according to claim 38, wherein the methacrylates have the following formula (II)
X—OC(O)C(CH₂)(CH₃) (II),
wherein X is selected from the group consisting of C₁-C₂₄ alkyl, [(CH₂)ₘO]ᵣ, C₂-C₂₀ alkenyl, vinyl and allyl, wherein r is an integer from 1 to 10.

44. The nanocomposite material according to claim 43, wherein the methacrylates are selected from the group consisting of di(propylene glycol) allyl ether methacrylate, propargyl methacrylate, 2-(methacryloyloxy)ethyl ester, allyl methacrylate, allyl acrylate, propargyl methacrylate and propargyl acrylate.

45. The nanocomposite material according to claim 38, wherein the acrylates have the following formula (III)
X—OC(O)C(CH₂)(H) (III),
wherein X is selected from the group consisting of C₁-C₂₄ alkyl, [(CH₂)ₘO]ᵣ, C₂-C₂₀ alkenyl, vinyl and allyl, wherein r is an integer from 1 to 10.

46. The nanocomposite material according to claim 45, wherein the acrylates are selected from the group consisting of acrylate, propargyl acrylate, allyl acrylate, 2-allyloxyethyl acrylate, 2-propargyloxyethyl acrylate and 1-hexenylacrylate.

47. The nanocomposite material according to claim 38, wherein the epoxides are selected from the group consisting of 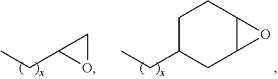
wherein x is an integer from 1 to 10, 3,4-epoxycyclohexylmethyl-3,4-epoxycyclohexanecarboxylate, 3,4-epoxy-2-methylcyclohexylmethyl-3,4-epoxy-2-methylcyclohexane carboxylate, bis(3,4-epoxy-6-methylcyclohexylmethyl) adipate, 4-vinyl-cyclohexane epoxide.

48. The nanocomposite material according to claim 38, wherein a is n.

49. The nanocomposite material according to claim 38, wherein a is n−2 and b is 2.

50. The nanocomposite material according to claim 38, wherein a and b are the same.

51. The nanocomposite material according to claim 48, wherein n is 8.

52. The nanocomposite material according to claim 48, wherein R¹ and R² are independently selected from di(propylene glycol) allyl ether methacrylate, 4-vinyl-cyclohexene epoxide and propargyl methacrylate.

53. The nanocomposite material according to claim 38, wherein the polymerizable monomers further comprise bisphenylglycidyl methacrylate (Bis-GMA), triethylene glycol dimethacrylate (TEGDMA), urethane dimethacrylate (UDMA), ethoxylated bisphenol-A-dimethacrylate (bis-EMA), decanediol dimethacrylate (D₃MA), urethane tetramethacrylate (UTMA), methyl methacrylate, 2-hydroxyethyl methacrylate, hexandiol methacrylate, dodecanediol dimethacrylate, bisphenol-A-dimethacrylate, 2,6-di-tert-butyl-4-metyhlphenol (BHT), 2-hydroxyethylmethacrylate (HEMA), N,N-dimethyl-p-toluidine or mixtures thereof.

54. The nanocomposite material according to claim 38, wherein the curing system is selected from the group consisting of polymerization initiators, polymerization accelerators, stabilizers, ultraviolet light absorbers, cationic initiators and antioxidants.

55. The nanocomposite material according to claim 54, wherein the polymerization accelerator is selected from the group consisting of amine, ammonia and an acid.

56. The nanocomposite material according to claim 55, wherein the amine is a tertiary amine selected from the group consisting of N(C₂H₅)₃, N(C₆H₅)₃, ethyl 4-(dimethylamino)benzoate (EDMAB), 2-[4-(dimethylamino)phenyl]ethanol, N,N-dimethyl-p-toluidine (DMPT), bis(hydroxyethyl)-p-toluidine, dimethylaminoethyl methacrylate and diethylaminoethyl methacrylate (DEAEMA).

57. The nanocomposite material according to claim 38, wherein the at least one filler is selected from the group consisting of silica, quartz, silica glass, strontium silicate, strontium borosilicate, lithium silicate, lithium alumina silicate, amorphous silica, ammoniated or deammoniated calcium phosphate, tricalcium phosphate alumina, zirconia, tin oxide, titania, apatites, hydroxyapatites, modified hydroxyapatite compositions, bismuth oxide, barium sulfate, bismuth subcarbonate or mixtures thereof.

58. The nanocomposite material according to claim 57, wherein the silica glass comprises strontium, barium, zinc, boron, yttrium, aluminoborosilicate glass, strontium-alumino-fluoro-silicate glass, colloidal glass or other fluoride releasing glasses.

59. The nanocomposite material according to claim 38 further comprising a therapeutic agent.

60. The nanocomposite material according to claim 59, wherein the therapeutic agent is an antibacterial agent and/or a remineralization agent.

61. The nanocomposite material according to claim 59, wherein the therapeutic agent is selected from the group consisting of calcium compounds, fluorides, sodium and potassium monofluorophosphate, zinc compounds, chlorohexidine, halogenated diphenyl ether and phenolic antibacterial compounds.

62. The nanocomposite material according to claim 39, wherein the SSQ is used in an amount of about 20 wt % based on the total amount of the nanocomposite material.

63. The nanocomposite material according to claim 38 being a low-shrinking material.

64. The nanocomposite material according to claim 63, wherein the material has a linear percent shrinkage below about 4.0.

65. The nanocomposite material according to claim 64, wherein the material has a linear percent shrinkage below about 2.0.

66. The nanocomposite material according to claim 65, wherein the material has a linear percent shrinkage in the range of about 0.25 to about 0.5.

67. A method of preparing a polymerizable resin for a biologically compatible material comprising contacting a nanocomposite material according to claim 38 with the biologically compatible material.

68. The method according to claim 67, further comprises applying the nanocomposite material to a site of a tooth and subsequently curing the nanocomposite material.

69. The method according to claim 67, wherein the polymerizable resin is a coating, lens or plastic.

70. The nanocomposite material according to claim 54 wherein the cationic initiators is incorporated to the nanocomposite to allow for a dual cure system where the epoxy ring can be opened to compensate for volumetric shrinkage for the multi-functionalized SSQ.
